(19) United States
(12) Patent Application Publication
(10) Pub. No.: US 2013/0323284 A1
(43) Pub. Date: Dec. 5, 2013

(54) USE OF mTOR INHIBITORS TO TREAT BACTERIAL INFECTION

(71) Applicant: Jose A. Bengoechea Alonso, Bunyola (ES)

(72) Inventor: Jose A. Bengoechea Alonso, Bunyola (ES)

(73) Assignee: JAZZYA INVESTMENTS, Madrid (ES)

(21) Appl. No.: 13/488,783

(22) Filed: Jun. 5, 2012

(51) Int. Cl. A61K 31/436; C07D 487/18; A61P 37/04; A61P 31/00; A61P 29/00; A61P 31/04; A61K 31/713; C07H 21/02

(52) U.S. Cl. 424/278.1; 514/291; 514/44 A; 540/456; 536/24.5

(57) ABSTRACT

A method for treating a mammal having an infection with an organism that persists intracellularly in a mammalian cell by inducing an autophagy defect includes administering to the mammal an effective dose of a bioavailable agent that restores autophagy function, by inhibition of the mTOR pathway.

### BACKGROUND OF THE INVENTION

1. Field of the Invention

The present invention relates to the field of method and compositions for treating bacterial infections with organisms that persists intracellularly.

2. Discussion of Related Art

Infectious diseases are leading causes of human morbidity and mortality. Chiefly, lower respiratory tract infections (not included tuberculosis and HIV/AIDS-associated pneumonia) and bacteria-caused diarrhea account for more than 12% of the global burden of disease [141] [142]. This disease burden is greater than those of other better recognize causes of disease such as malaria, cancer or heart attacks [141] [142]. The main strategy for fighting infectious diseases has focused on targeting enzymes from pathogens with antibiotics and other antimicrobials. Challenged by decades of drug exposure, bacteria have evolved defensive mechanisms to render commonly used antimicrobials ineffective. Thus, the growing number of organisms resistant to currently available antibiotics has become a major public threat worldwide. The rapid development of resistance shortens the life span of a therapeutic agent, leading to decreased interest of the industry to develop new agents because the costs are prohibitive compared to the economic potential of the drug. Therefore, there is a need to develop effective therapeutics based on new targets/approaches.

The Gram negative human pathogen Klebsiella pneumoniae causes a wide range of infections, from urinary tract infections to pneumonia. The latter is particularly devastating among immunocompromised patients with mortality rates between 25% and 60%3. In addition, Klebsiella pneumoniae is one of the most frequent antibiotic-resistant bacteria isolated in hospitals but also in the community. The isolation worldwide of Klebsiella pneumoniae strains already resistant to carbapenemes and colistin, the last antibiotic hope, makes many Klebsiella infections virtually untreatable with the available drugs.

A substantial amount of research over the last 20 years has focused on the importance of inflammatory responses in the host defense against Klebsiella pneumoniae pulmonary infection. Nearly all these studies have examined infection of a wild-type strain after intratracheal inoculation and compared outcomes in both wild-type and immunodeficient mice. These studies have been carried out mainly by the groups of Theodore Standiford (University of Michigan, USA) and Jay Kolls (Louisiana State University, USA). The information obtained indicates that activation of inflammatory responses is essential to clear Klebsiella pneumoniae infections [144] [145] [146] and that the germ-line encoded “Toll-like” receptors (TLRs) play a major role in detecting Klebsiella pneumoniae [147] [148]. Conversely, this suggests that Klebsiella pneumoniae tries to counteract the induction of these host defense responses. Indeed, it has been shown [149] [150] [151] that, in sharp contrast to wild-type strains, avirulent Klebsiella pneumoniae mutants activate an inflammatory program through TLR-dependent pathways. Therefore, Klebsiella pneumoniae pathogenesis may be associated with its ability to modulate the innate immune system in its own benefit.

Recently, Klebsiella pneumoniae has been demonstrated to reduce the activation of the main cellular signaling pathways, AP-1 and NF-κB pathways, which the host turns on upon infection to activate an inflammatory defense response [152]. When infecting human airway epithelial cells, Klebsiella pneumoniae inhibits the cytokine-dependent nuclear translocation of NF-κB by affecting the ubiquitination status of key intermediates of the signaling pathway in a process dependent on the activation of the deubiquitinase CYLD (see also FIG. 1). Klebsiella pneumoniae also targets the phosphorylation status of p38, ERK and JNK MAP kinases by activating the expression of a specific phosphatase, MKP-1. Importantly, data of the present inventors demonstrated that Klebsiella pneumoniae induces the expression of CYLD and MKP-1 in the lungs of infected mice [149, 152].

Mammalian target of rapamycin (mTOR) is a serine/threonine protein kinase known to play a role in regulating cell growth, cell proliferation, cell motility, cell survival, protein synthesis and transcription. Dysregulation of the mTOR pathway is implicated as a contributing factor to various human diseases, particularly various types of cancer. Rapamycin is a natural product produced by the bacterium Streptomyces hygroscopicus that can inhibit mTOR through association with its intracellular receptor FK-506 binding protein 12 (FKBP12). The FKBP12-rapamycin complex binds directly to the FKBP12-rapamycin binding domain of mTOR. mTOR functions as a catalytic subunit for two distinct molecular complexes, mTOR complex 1 (mTORC1) and mTOR complex 2 (mTORC2). In addition to mTOR, mTORC1 is composed of regulatory associated protein of mTOR (Raptor) and mammalian LST8/G-protein β-subunit like protein (mLST8/GβL). This complex functions as a nutrient/energy/redox sensor and plays a role in regulating protein synthesis. The activity of mTORC1 is stimulated by insulin, growth factors, serum, phosphatidic acid, amino acids (particularly leucine) and oxidative stress (Hay and Sonenberg, Genes Dev. 18(16):1926-1945, 2004; Wullschleger et al., Cell 124(3):471-484). In contrast, mTORC1 is known to be inhibited by low nutrient levels, growth factor deprivation, reductive stress, caffeine, rapamycin, farnesylthiosalicylic acid and curcumin (Beevers et al., Int. J. Cancer 119(4):757-764, 2006; McMahon et al., Mol. Endocrinol. 19(1):175-183). The components of mTORC2 are rapamycin-insensitive companion of mTOR (Rictor), GβL, mammalian stress-activated protein kinase interacting protein 1 and mTOR. mTORC2 has been shown to function as an important regulator of the cytoskeleton through its stimulation of F-actin stress fibers, paxillin, RhoA, Rac1, Cdc42 and protein kinase C alpha (Sarbassov et al., Curr. Biol. 14(14): 1296-302, 2004; Sarbassov et al., Science 307(5712): 1098-101, 2005). Unlike mTORC1, mTORC2 is not sensitive to rapamycin.

A number of mTOR inhibitors are currently being used, or are currently being investigated in clinical trials, to treat a variety of conditions Inhibitors of mTOR, such as rapamycin, are known to exhibit immunosuppressive and anti-proliferative properties. Accordingly, mTOR inhibitors are routinely administered to transplant recipients to prevent organ or bone marrow rejection.

Rapamycin (sirolimus)

((3S,6R,7E,9R,10R,12R,14S,15E,17E,19E,21S,23S,26R,27R,34aS)-9,10,12,13,14,-21,22,23,24,25,26,27,32,33,34,34a-hexadecahydro-9,27-dihydroxy-3-[(1R)-2-[-(1S,3R,4R)-4-hydroxy-3-methoxycyclohexyl]-1 -methylethyl]-10,21-dimethoxy-6-,8,12,14,20,26-hexamethyl-23,27-epoxy-3H-pyrido[2,1-c] [1,4]-oxaazacyclohen-triacontine-1,5,11,28,29(4H,6H,31H)-pentone), the prototype mTOR inhibitor, is used clinically in renal transplant to prevent rejection, and shows potent immunosuppressive and anti-tumor activities.

Rapamycin and related drugs administered as immunosuppressants for renal transplant patients has been associated with Klebsiella pneumoniae infection. Thus, the literature suggests that Rapamycin administration is part of the etiology of the infection.

Other mTOR inhibitors include everolimus, tacrolimus, zotarolimus (ABT-578), pimecrolimus, biolimus, FK-506, PP242 (2-(4-Amino-1-isopropyl-1H-pyrazolo[3,4-d]pyrimidin-3-yl)-1H-indol-5-ol), Ku-0063794 (re1-5-[2-[(2R,6S)-2,6-Dimethyl-4-morpholinyl]-4-(4-morpholinyl)pyrido[2,3-d]pyrimidin-7-yl]-2-methoxybenzenemethanol), PI-103 (3-(4-(4-Morpholinyl)pyrido[3′,2′:4,5]furo[3,2-d]pyrimidin-2-yl)phenol), PKI-179 (N-[4-[4-(4-Morpholinyl)-6-(3-oxa-8-azabicyclo[3.2.1]oct-8-yl)-1,3,5-triazin-2-yl]phenyl]-N′-4-pyridinylurea hydrochloride), AZD8055 (5-[2,4-Bis[(3S)-3-methyl-4-morpholinyl]pyrido[2,3-d]pyrimidin-7-yl]-2-methoxybenzenemethanol), WYE-132/WYE-125132 (1-{4-[1-(1,4-Dioxa-spiro[4.5]dec-8-yl)-4-(8-oxa-3-aza-bicyclo[3.2.1]oct-3-yl)-1H-pyrazolo[3,4-d]pyrimidin-6-yl]-phenyl}-3-methyl-urea), WYE-23 (4-{6-[4-(3-Cyclopropyl-ureido)-phenyl]-4-morpholin-4-yl-pyrazolo[3,4-d]pyrimidin-1-yl}-piperidine-1-carboxylic acid methyl ester), WYE-28 (4-(6-{4-[3-(4-Hydroxymethyl-phenyl)-ureido]-phenyl}-4-morpholin-4-yl-pyrazolo[3,4-d]pyrimidin-1-yl)-piperidine-1-carboxylic acid methyl ester), WYE-354 (4-[6-[4-[(Methoxycarbonyl)amino]phenyl]-4-(4-morpholinyl)-1H-pyrazolo[3,4-d]pyrimidin-1-yl]-1piperidinecarboxylic acid methyl ester), C20-methallylrapamycin and C16-(S)-butylsulfonamidorapamycin, C16-(S)-3-methylindolerapamycin (C16-iRap), C16-(S)-7-methylindolerapamycin (AP21967/C16-AiRap), CCI-779 (temsirolimus), RAD001(40-O-(2-hydroxyethyl)-rapamycin), AP-23575, AP-23675, AP-23573, 20-thiarapamycin, 15-deoxo-19-sulfoxylrapamycin, WYE-592, ILS-920, (3S,6R,7E,9R,10R,12R,14S,15E,17E,19E,21S,23S,26R,27R,34aS)-9,10,12,13,14,2-1,22,23,24,25,26,27,32,33,34,34a-Hexadecahydro-9,27-dihydroxy-3-[(1R)-2-[(-1S,3R,4R)-3-methoxy-4-tetrazol-1-yl)cyclohexyl]-1-methylethyl]-10,21-dime-t-hoxy-6,8,12,14,20,26-hexamethyl-23,27-epoxy-3H-pyrido[2,1-c] [1,4]oxaazac-yc-lohentriacontine-1,5,11,28,29(4H,6H,31H)-pentone) 23,27-Epoxy-3H pyrido [2,1-c] [1,4]oxaazacyclohentriacontine-1,5,11,28,29(4H,6H,31H)-pentone (U.S. Pat. No. 6,015,815), U.S. Pat. No. 6,329,386, U.S. Publication 2003/129215, U.S. Publication 2002/123505, A-94507, Deforolimus, AP-23675, AP-23841, Zotarolimus, CCI779/Temsirolimus, RAD-001/Everolimus, 7-epi-rapamycin, 7-thiomethyl-rapamycin, 7-epi-trimethoxy-rapamycin, 2-desmethyl-rapamycin, and 42-O-(2-hydroxy)ethyl-rapamycin, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, 42-O-(2-hydroxy)ethyl rapamycin, ridaforolimus, ABI-009, MK8669, TOP216, TAFA93, TORISEL™ (prodrug), CERTICAN™, Ku-0063794, PP30, Torin1, ECO371, AP23102, AP23573, AP23464, AP23841; 40-(2-hydroxyethyl)rapamycin, 40-[3-hydroxy(hydroxymethyl)methylpropanoate]-rapamycin (also called CC1779), 32-deoxorapamycin, and 16-pentynyloxy-32(S)-dihydrorapanycin.

Further rapamycin analogs include rapamycin oximes (U.S. Pat. No. 5,446,048); rapamycin aminoesters (U.S. Pat. No. 5,130,307); rapamycin dialdehydes (U.S. Pat. No. 6,680,330); rapamycin 29-enols (U.S. Pat. No. 6,677,357); O-alkylated rapamycin derivatives (U.S. Pat. No. 6,440,990); water soluble rapamycin esters (U.S. Pat. No. 5,955,457); alkylated rapamycin derivatives (U.S. Pat. No. 5,922,730); rapamycin amidino carbamates (U.S. Pat. No. 5,637,590); biotin esters of rapamycin (U.S. Pat. No. 5,504,091); carbamates of rapamycin (U.S. Pat. No. 5,567,709); rapamycin hydroxyesters (U.S. Pat. No. 5,362,718); rapamycin 42-sulfonates and 42-(N-carbalkoxy)sulfamates (U.S. Pat. No. 5,346,893); rapamycin oxepane isomers (U.S. Pat. No. 5,344,833); imidazolidyl rapamycin derivatives (U.S. Pat. No. 5,310,903); rapamycin alkoxyesters (U.S. Pat. No. 5,233,036); rapamycin pyrazoles (U.S. Pat. No. 5,164,399); acyl derivatives of rapamycin (U.S. Pat. No. 4,316,885); reduction products of rapamycin (U.S. Pat. Nos. 5,102,876 and 5,138,051); rapamycin amide esters (U.S. Pat. No. 5,118,677); rapamycin fluorinated esters (U.S. Pat. No. 5,100,883); rapamycin acetals (U.S. Pat. No. 5,151,413); oxorapamycins (U.S. Pat. No. 6,399,625); and rapamycin silyl ethers (U.S. Pat. No. 5,120,842), U.S. Pat. No. 7,153,957 (Regioselective synthesis of CCI-779), U.S. Pat. No. 7,122,361 (Compositions employing a novel human kinase), U.S. Pat. No. 7,105,328 (Methods for screening for compounds that modulate pd-1 signaling), U.S. Pat. No. 7,074,804 (CCI-779 Isomer C), U.S. Pat. No. 7,060,797 (Composition and method for treating lupus nephritis), U.S. Pat. No. 7,060,709 (Method of treating hepatic fibrosis), U.S. Pat. No. 7,029,674 (Methods for downmodulating immune cells using an antibody to PD-1), U.S. Pat. No. 7,019,014 (Process for producing anticancer agent LL-D45042), U.S. Pat. No. 6,958,153 (Skin penetration enhancing components), U.S. Pat. No. 6,821,731 (Expression analysis of FKBP nucleic acids and polypeptides useful in the diagnosis of prostate cancer), U.S. Pat. No. 6,713,607 (Effector proteins of Rapamycin), U.S. Pat. No. 6,670,355 (Method of treating cardiovascular disease), U.S. Pat. No. 6,617,333 (Antineoplastic combinations), U.S. Pat. No. 6,541,612 (Monoclonal antibodies obtained using rapamycin position 27 conjugates as an immunogen), U.S. Pat. No. 6,511,986 (Method of treating estrogen receptor positive carcinoma), U.S. Pat. No. 6,440,991 (Ethers of 7-desmethylrapamycin), U.S. Pat. No. 6,432,973 (Water soluble rapamycin esters), U.S. Pat. No. 6,399,626 (Hydroxyesters of 7-desmethylrapamycin), and U.S. Pat. No. 6,399,625 (1-oxorapamycins), each of which is specifically incorporated by reference. Numerous chemical modifications of rapamycin have been attempted. These include the preparation of mono- and di-ester derivatives of rapamycin (WO 92/05179), 27-oximes of rapamycin (EPO 467606); 42-oxo analog of rapamycin (U.S. Pat. No. 5,023,262); bicyclic rapamycins (U.S. Pat. No. 5,120,725); rapamycin dimers (U.S. Pat. No. 5,120,727); silyl ethers of rapamycin (U.S. Pat. No. 5,120,842); and arylsulfonates and sulfamates (U.S. Pat. No. 5,177,203).

Other analogs of rapamycin include those described in U.S. Pat. Nos. 8,134,344; 8,034,926; 8,008,318; 7,897,608; 7,820,812; 7,795,252; 7,560,457; 7,538,119; 7,476,678; 7,470,682; 7,455,853; 7,446,111; 7,445,916; 7,282,505; 7,279,562; 7,273,874; 7,268,144; 7,241,771; 7,220,755; 7,160,867; 7,091,213, 6,329,386; RE37,421; U.S. Pat. Nos. 6,200,985; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,258,389; 5,256,790; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; 5,023,262; 20120064143; 20120028908; 20110230515; 20110129496; 20110009618; 20110009403; 20110009325; 20100260733; 20100248265; 20100233733; 20100104626; 20100081681; 20090149511; 20090148859; 20080249123; 20080188511; 20080182867; 20080091008; 20080085880; 20080069797; 20070280992; 20070225313; 20070203172; 20070203171; 20070203170; 20070203169; 20070203168; 20070142423; 20060264453; 20040010002; WO 98/02441; WO 01/14387; WO/05005434;; WO 94/090101; WO 92/05179; WO 93/111130; WO 94/02136; WO 94/02485; WO 95/14023; WO 94/02136; WO 95/16691; WO 96/41807; WO 96/41807; WO/05016252; WO96/41865; WO 99/36553; WO 01/14387; WO 2007/135411; WO 98/02441; WO 01/14387; WO 03/64383; U.S. Provisional Application No. 60/528,340, EP1880723; each of which is expressly incorporated herein by reference. See also Rivera et al, Proc Natl Acad Sci USA 96, 8657 8662; Ye, X. et al (1999) Science 283, 88 91, Yu, K. et al., Endocrine-Related Cancer (2001) 8, 249 258; Geoerger, B. et al., Cancer Res. (2001) 61 1527 1532); Dancey, Hematol Oncol Clin N Am 16 (2002):1101 1114, each of which is expressly incorporated herein by reference. Information concerning rapamycin synthesis can be found in Schwecke et al., 1995; Gregory et al., 2004; Gregory et al., 2006; and Graziani, 2009.

Non-rapamycin analog mTOR inhibiting compounds include, but are not limited to, LY294002, wortmannin, quercetin, myricentin, staurosporine, and ATP competitive inhibitors (see U.S. patent application Ser. Nos. 11/361,213 and 11/361,599, each of which are incorporated by references herein in their entirety).

The mammalian target of rapamycin (mTOR) also known as mechanistic target of rapamycin or FK506 binding protein 12-rapamycin associated protein 1 (FRAP1) is a protein which in humans is encoded by the FRAP1 gene. [1] [2] mTOR is a serine/threonine protein kinase that regulates cell growth, cell proliferation, cell motility, cell survival, protein synthesis, and transcription. [3] [4] mTOR belongs to the phosphatidylinositol 3-kinase-related kinase protein family. See, en.wikipedia.org/wiki/Mammalian_target_of_rapamycin (May 31, 2012), expressly incorporated herein by reference.

mTOR integrates the input from upstream pathways, including insulin, growth factors (such as IGF-1 and IGF-2), and amino acids. [3] mTOR also senses cellular nutrient and energy levels and redox status. [5] The mTOR pathway is dysregulated in human diseases, especially certain cancers. [4] Rapamycin is a bacterial product that can inhibit mTOR by associating with its intracellular receptor FKBP12. [6] [7] The FKBP12-rapamycin complex binds directly to the FKBP12-Rapamycin Binding (FRB) domain of mTOR. [7] mTOR is the catalytic subunit of two molecular complexes. [8]

mTOR stands for mammalian Target Of Rapamycin and was named based on the precedent that TOR was first discovered via genetic and molecular studies of rapamycin-resistant mutants of Saccharomyces cerevisiae that identified FKBP12, Tor1, and Tor2 as the targets of rapamycin and provided robust support that the FKBP12-rapamycin complex binds to and inhibits the cellular functions of Tor1 and Tor2.

mTOR Complex 1 (mTORC1) is composed of mTOR, regulatory-associated protein of mTOR (Raptor), mammalian LST8/G-protein [3-subunit like protein (mLST8/GβL) and the recently identified partners PRAS40 and DEPTOR. [9] [10] This complex is characterized by the classic features of mTOR by functioning as a nutrient/energy/redox sensor and controlling protein synthesis. [3] [9] The activity of this complex is stimulated by insulin, growth factors, serum, phosphatidic acid, amino acids (particularly leucine), and oxidative stress. [9] [11]

mTORC1 integrates four major signal inputs: nutrients, growth factors, energy and stress. Amino acids are imported into the cell by amino acid transporters. The presence of amino acids causes Rag GTPases heterodimers to switch to its active conformation. Active Rag heterodimers interact with RAPTOR, localizing mTORC1 to the surface of late endosomes and lysosome where the Rag GTPases are located. [12] This allows mTORC1 to physically interact with RHEB, which is activated by growth factors such as insulin. [13] Thus, nutrient and growth factor signals are integrated at this point where both inputs are required for mTORC1 activation. RHEB has an essential role in mTORC1 signaling in that its loss prevents activation of mTORC1, while its overexpression can maintain mTORC1 activity when nutrients and growth factor have been withdrawn. [14] Growth factors such as insulin regulate the GTP loading of RHEB by activating the PI3K pathway which leads to the phosphorylation and activation of Akt. [15] In turn, Akt phosphorylates TSC2, which is part of the TSC1-TSC2 complex that acts as a GAP for RHEB. [15] TSC-2 phosphorylation by Akt inhibts its GAP activity for RHEB, promoting mTORC1 activation. Akt also phosphorylates PRAS40, preventing it from inhibiting mTORC 1. Growth factors can also signal the ERK and Wnt pathway to activate mTORC1. [16] The mTORC1 pathway also senses energy through the AMP-activated kinase (AMPK). When the AMP:ATP ratio increases, AMPK phosphorylates TSC2 and RAPTOR, leading to inhibition of mTORC1. [17] Various stressors including hypoxia and DNA damage can also inhibit mTORC1. [18]

The two best characterized targets of mTORC1 are p70-S6 Kinase 1 (S6K1) and 4E-BP1, the eukaryotic initiation factor 4E (eIF4E) binding protein 1. [3] mTORC1 phosphorylates S6K1 on at least two residues, with the most critical modification occurring on a threonine residue (T389). [19] [20] This event stimulates the subsequent phosphorylation of S6K1 by PDK1. [20] [21] Active S6K1 can in turn stimulate the initiation of protein synthesis through activation of S6 Ribosomal protein (a component of the ribosome) and other components of the translational machinery. [22] S6K1 can also participate in a positive feedback loop with mTORC1 by phosphorylating mTOR's negative regulatory domain at two sites; phosphorylation at these sites appears to stimulate mTOR activity. [23] [24]

mTORC1 has been shown to phosphorylate at least four residues of 4E-BP1 in a hierarchical manner. [6] [25] [26] Non-phosphorylated 4E-BP1 binds tightly to the translation initiation factor eIF4E, preventing it from binding to 5′-capped mRNAs and recruiting them to the ribosomal initiation complex. [27] Upon phosphorylation by mTORC1, 4E-BP1 releases eIF4E, allowing it to perform its function. [27] The activity of mTORC1 appears to be regulated through a dynamic interaction between mTOR and Raptor, one which is mediated by GβL. [9] [10] Raptor and mTOR share a strong N-terminal interaction and a weaker C-terminal interaction near mTOR's kinase domain. [9] When stimulatory signals are sensed, such as high nutrient/energy levels, the mTOR-Raptor C-terminal interaction is weakened and possibly completely lost, allowing mTOR kinase activity to be turned on. When stimulatory signals are withdrawn, such as low nutrient levels, the mTOR-Raptor C-terminal interaction is strengthened, essentially shutting off kinase function of mTOR. [9]

mTOR Complex 2 (mTORC2) is composed of mTOR, rapamycin-insensitive companion of mTOR (Rictor), GβL, and mammalian stress-activated protein kinase interacting protein 1 (mSIN1). [28] [29] mTORC2 has been shown to function as an important regulator of the cytoskeleton through its stimulation of F-actin stress fibers, paxillin, RhoA, Rac1, Cdc42, and protein kinase C α (PKCα). [29] mTORC2 also appears to possess the activity of a previously elusive protein known as “PDK2”. mTORC2 phosphorylates the serine/threonine protein kinase Akt/PKB at a serine residue S473. Phosphorylation of the serine stimulates Akt phosphorylation at a threonine T308 residue by PDK1 and leads to full Akt activation; [30] [31] curcumin inhibits both by preventing phosphorylation of the serine. [4]

mTORC2 appears to be regulated by insulin, growth factors, serum, and nutrient levels. [28] Originally, mTORC2 was identified as a rapamycin-insensitive entity, as acute exposure to rapamycin did not affect mTORC2 activity or Akt phosphorylation. [30] However, subsequent studies have shown that, at least in some cell lines, chronic exposure to rapamycin, while not affecting pre-existing mTORC2s, promotes rapamycin inhibition of free mTOR molecules, thus inhibiting the formation of new mTORC2. [32]

Rapamycin inhibits mTORC1, and this appears to provide most of the previously reported beneficial effects of the drug (including life-lengthening in animal studies). Rapamycin also acts on mTORC2. Disruption of mTORC2 produces diabetic-like symptoms of decreased glucose tolerance and insensitivity to insulin also associated with rapamycin. [33]

The mTORC2 signaling pathway is less clearly defined than the mTORC1 signaling pathway. Therefore, performing knockout experiments is a good way to shed light on more specific molecules and their roles in this pathway. Protein function inhibition using knockdowns and knockouts were found to produce the following phenotypes: NIP7: knockdown reduced mTORC2 activity which is indicated by decreased phosphorylation of mTORC2 substrates. [34]; RICTOR: overexpression leads to metastasis and knockdown inhibits growth factor induced PKC-phosphorylation. [35]; mTOR: inhibition of mTORC1 and mTORC2 by PP242 [2-(4-Amino-1-isopropyl-1H-pyrazolo[3,4-d]pyrimidin-3-yl)-1H-indol-5-ol] leads to autophagy or apoptosis; inhibition of mTORC2 alone by PP242 prevents phosphorylation of Ser-473 site on AKT and arrests the cells in G1 phase of the cell cycle.[36]; PDK1: knockout is lethal; hypomorphic allele results in smaller organ volume and organism size, but normal AKT activation. [37]; AKT: knockout mice experience spontaneous apoptosis (AKT1), severe diabetes (AKT2), small brains (AKT3), and growth deficiency (AKT1/AKT2) [38]mTOR inhibitors, e.g. rapamycin, are already used to prevent transplant rejection. Rapamycin is also related to the therapy of glycogen storage disease (GSD). Some articles reported that rapamycin can inhibit mTORC1 so that the phosphorylation of GS (glycogen storage) can be increased in skeletal muscle. This discovery represents a potential novel therapeutic approach for glycogen storage diseases that involve glycogen accumulation in muscle. Various natural compounds, including epigallocatechin gallate (EGCG), caffeine, curcumin, and resveratrol, have also been reported to inhibit mTOR when applied to isolated cells in culture; [4] [77] however, there is as yet no evidence that these substances inhibit mTOR when taken as dietary supplements. Some (e.g. temsirolimus, everolimus) are beginning to be used in the treatment of cancer. [78] [79] mTOR inhibitors may also be useful for treating several age-associated diseases. [80] Ridaforolimus is another mTOR inhibitor, currently in clinical development.

Mammalian target of rapamycin has been shown to interact with:[81]; Ab1 gene, [82]; AKT1, [83] [84] [85]; CLIP1, [86]; EIF3F[87]; EIF4EBP1, [88] [89] [90] [91] [92] [93] [94] [95]; FKBP1A, [96] [97] [98] [99] [100] [101]; GPHN, [102]; KIAA1303, [88] [89] [90] [91] [96] [97] [103] [104] [105] [106] [107] [108] [109] [110] [111] [112] [113] [114] [115] [116]; P70-S6 Kinase 1, [89] [91] [92] [93] [94] [111] [115] [117] [118] [119] [120] [121] [121] [122] [123] [124]; PRKCD, [125]; RHEB, [92] [126] [127] [128]; RICTOR, [96] [97] [104] [106] [113] [115] [116]; STAT1, [129]; STAT3, [130] [131] and; UBQLN1. [132]

U.S. Pat. No. 7,771,751 (Abraxis Bioscience, LLC) discloses Rapamycin (sirolimus) as an antibiotic or anti-cancer agent that is poorly water soluble, and is discussed as part of a formulation. It lists Klebsiella as a possible target of generic formulations, but not that rapamycin has any effect on Klebsiella.

U.S. Pat. No. 7,947,741 (Mpex Pharmaceuticals, Inc.) relates to the use of pentamidine and analogous compositions as efflux pump inhibitors to be co-administered with antimicrobial agents for the treatment of infections caused by drug resistant pathogens. The invention also includes compounds useful as efflux pump inhibitors and compositions and devices comprising an efflux pump inhibitor and an antimicrobial agent. A listed one, of many, target organisms is Klebsiella pneumonia. Rapamycin is mentioned as an antifungal agent, and as having a possible efflux pump inhibitory activity.

2010/0152098 (Mpex Pharmaceuticals Inc.) relates to polybasic bacterial efflux pump inhibitors and therapeutic uses thereof. This reference discusses Klebsiella pneumoniae as one of a number of pathogenic organisms. Similarly to U.S. Pat. No. 7,947,741, it states with respect to Rapamycin that it is an antifungal agent and possible efflux pump inhibitor. WO 2008141012 (Mpex Pharmaceuticals Inc.) is similar in disclosure to U.S. Pat. No. 7,947,741 and US 2010/0152098..

2006/0211752 (Kohn, et al.) relates to “Use of Phenylmethimazoles, Methimazole derivatives, and Tautomeric Cyclic Thiones for the Treatment of Autoimmune/Inflammatory Diseases Associated with Toll-Like Receptor Overexpression”. This reference discusses Klebsiella pneumonia as an example of an infectious disease. The compounds are discussed as being used with Rapamycin as an immunosuppressant agent that is compatible with other agents.

2010/0330111 (Sena) relates to compounds consisting of glycolipids covalently bound to an antigen or a drug via a linker. They induce a stronger immune response than a composition comprising separated glycolipids and antigen. The compounds are also able to target drug to CD1d restricted cells. One possible compound to be linked to the glycolipid is rapamycin. The antigen can be a Klebsiella antigen.

2011/0129496 (Ahmed, et al.) relates to a method of using mTOR Inhibitors to Enhance T Cell Immune Responses. Treatment of a subject with an mTOR inhibitor enhances antigen-specific T cell immune responses. The antigen can be any antigen, such as an antigen from a pathogen or a vaccine, or a tumor antigen. The mTOR inhibitor can be administered either before or after vaccination to enhance the quantity and quality of the T cell immune response and immunological memory. In some examples, the mTOR inhibitor is rapamycin or a rapamycin analog. (Abstract). The bacterial pathogen may be Klebsiella pneumoniae.

D C O Massey, M Parkes, “Genome-wide association scanning highlights two autophagy genes, ATG16L1 and IRGM, as being significantly associated with Crohn's disease”, Autophagy 3:6, 649-651; November/December 2007; 2007 mentions Klebsiella pneumoniae and Rapamycin, but in relation to Crohn's Disease. See also, Ivana R. Ferrer et al., “Cutting Edge: Rapamycin Augments Pathogen-Specific but Not Graft-Reactive CD8+T Cell Responses”, The Journal of Immunology Aug. 15, 2010 vol. 185 no. 4 2004-2008, Published online before print Jul. 14, 2010, doi: 10.4049/j immuno1.1001176.

### SUMMARY OF THE INVENTION

The present technology provides, for example, a pharmacologically effective dose of an mTOR inhibitor, for example Rapamycin or an analog thereof, as well as molecules modulating the activation status of the pathway, to treat an infection whose persistence is associated with a host autophagy defect. Another aspect of the technology provides a method for treating Klebsiella pneumoniae with an mTOR inhibitor to effectively enhance host cell autophagocytosis. A further aspect provides a method of using Rapamycin, Rapamycin analogs, and mTOR inhibitors, in the treatment of Klebsiella pneumoniae infections by inducing autophagocytosis. A further aspect provides a method of using Rapamycin, Rapamycin analogs, and mTOR inhibitors, in the treatment of Klebsiella pneumoniae infections by modulating the balance of pro-inflammatory anti-inflammatory cytokines towards pro-inflammatory cytokines.

The present technology provides, for example, a method for treating a mammal having an infection with an organism that persists intracellularly in a mammalian cell by inducing an autophagy defect and/or an anti-inflammatory milieu, by administering to the mammal an effective dose of an bioavailable agent which restores autophagy function and/or the inflammatory response, by inhibition of elements within the mTOR pathway. The bioavailable agent may be an mTOR inhibitor, or, for example, a protein kinase inhibitor of a kinase downstream of mTOR. The bioavailable agent may comprise rapamycin, or a derivative or analog thereof, as well as molecules modulating the activation status of the pathway.

Rapamycin and mTOR (mammalian target of Rapamycin) inhibitors according to the present technology differ from traditional antibiotics, in that they seek to modulate host cell response to infection by such organisms as Klebsiella pneumoniae which have as one characteristic that the host cells internalize of the bacterium, without killing it. Klebsiella pneumoniae activates mTOR, which leads to a failure of a proper immune response (including autophagy) to kill Klebsiella pneumoniae. Rapamycin, the prototype mTOR inhibitor, can block that pathway, and thus restore the host cell's ability to kill the Klebsiella pneumoniae. More generally, by restoring the cellular function(s) targeted by pathogens (e.g., Klebsiella pneumoniae) the cells will clear the pathogen.

Klebsiella pneumoniae exploits the cellular receptors NOD1, EGFR and the signaling cascade PI3K-mTOR to subvert the activation of host defense immune responses. The proposed therapeutic paradigm includes use of mTOR inhibitors (and other compositions that effectively block agonism of the mTOR pathway without undue host toxicity), however, the therapy may be directed to any appropriate target within the mTOR and related biochemical pathways, to achieve the same effect, a restoration of the autophagosomal capacity of the cells which internalize the bacteria as well as modulating the inflammatory response towards an effective host defense response. The present technology is not limited to inhibition of mTOR, and also encompasses the inhibition of the effect of agonism of various elements within the mTOR pathway (as discussed above) toward ineffective autophagy and/or activation of inflammatory responses of microorganisms such as Klebsiella.

Mouse testing has shown the anti-Klebsiella pneumoniae activity of Rapamycin.

It is therefore an object to provide a method for treating a mammal having an infection with an organism that persists intracellularly in a mammalian cell by at least one of reducing autophagy, inducing elevated levels of anti-inflammatory cytokines, and reducing levels of inflammatory cytokines, comprising administering to the mammal an therapeutic dose of a bioavailable agent which effectively increases autophagy function, by inhibition of at least one element within the mTOR pathway.

Another object provides a method of treating a mammal persistently infected with Klebsiella pneumoniae, comprising administering an effective amount in an effective regimen of an mTOR inhibitor to increase autophagocytic function of lung macrophages to kill the Klebsiella pneumoniae.

A further object provides a method of enhancing macrophage response in a subject having an infection with an intracellular organism that activates mTOR, in need of treatment, comprising administering to the subject a therapeutically effective amount of an mTOR inhibitor, thereby inhibiting mTOR and enhancing autophagocytic activity of the macrophage.

A still further object provides a pharmaceutically acceptable dosage form for administration to a human, comprising: a pharmaceutically acceptable inhibitor of an effect of an mTOR agonist on reducing autophagocytosis of a bacteria living within an autophagosome, in an effective amount and bioavailable form to increase an autophagocytic function of a mammalian cell to kill the bacteria.

The bioavailable agent may inhibit mTOR directly, or an upstream or downstream element within the pathway. For example, the bioavailable agent may also inhibit a protein kinase. The bioavailable agent may comprise rapamycin or a rapamycin analog. The bioavailable agent may also comprise a selective PI-3K inhibitor, or a selective EGFR inhibitor. The bioavailable agent is preferably effective for restoring NF-κB activation as part of a physiological inflammatory response in airway epithelial cells and alveolar macrophages which is selectively inhibited by Klebsiella pneumoniae infection.

Various compositions may be used in combination, either in amounts that are each effective to restore immune function, or in amounts that together lead to an effective response. Likewise, the mTOR pathway inhibitors may also be administered in conjunction with traditional therapies for the bacterial infection of the mammal to be treated, such as broad-spectrum antibiotics.

A bioavailable agent which inhibits the mTOR pathway and a broad spectrum antibiotic may be provided in a pharmaceutically acceptable dosage form comprising an oral dosage form which delivers an effective dose of the bioavailable agent and the broad spectrum antibiotic, and for example are administered in a course of therapy comprising 1 to 4 oral doses per day. The course of therapy may be weeks or months, and in the case of immunosuppressed patients, chronic administration may be appropriate.

The mTOR inhibitor may be provided in a pharmaceutically acceptable, orally bioavailable dosage form adapted for administration to an adult human in an efficacious dose.

The composition administered to the mammal or patient preferably achieves an effective level in lung tissue, and is preferably administered in such manner to effect lung macrophages.

### BRIEF DESCRIPTION OF THE DRAWINGS

FIG. 1 shows pathways manipulated by Klebsiella pneumoniae to block NF-κB

FIG. 2 shows an intracellular lifestyle of Klebsiella pneumoniae

FIG. 3 shows Production of i110 (upper panels) and i112 (lower panels) mRNA by infected macrophages for different time points (CON, non-infected cells; black bars, cells were treated with mTOR inhibitor before infection)

FIG. 4 shows mTOR inhibition reduces lung bacterial loads

FIG. 5 shows mTOR inhibition reduces Klebsiella pneumoniae intracellular survival.

FIGS. 6-12 show various illustrations of the mTOR signaling pathway

FIG. 13 shows a network of interactions of components of the PI3K, EGFR and ERK pathways.

FIG. 14 shows an immunoblot that shows that Klebsiella pneumoniae induces the phosphorylation of AKT, ERK and GSK3b in a PI3K-dependent manner.

FIG. 15 shows an immunoblot that shows that Klebsiella pneumoniae induces the production of CYLD in A PI3K-AKT-ERK dependent manner.

FIG. 16 is an immunoblot that shows that Klebsiella pneumoniae induces the phosphorylation of AKT, ERK and GSK3b in an EGFR-dependent manner.

### DESCRIPTION OF THE EMBODIMENT(S)

Rapamycin is an “antibiotic” better known as an immune modulator, i.e., for renal transplants. The present technology involves exploiting the host biology of Rapamycin to enhance host killing/clearance of Klebsiella pneumoniae organisms, and other organisms, such as Burkholderia, a pathogen associated with cystic fibrosis, that suppress or block autophagy and thus exploit similar host mechanisms for virulence. See, e.g., Abdulrahman BA et al 2011 Autophay 7:1359 (Burkholderia cenocepacia); Gutiérrez et al 2004 Cell 119:763 (Mycobacterium tuberculosis); Chiu et al 2009 Antimicrobial Agents and Chemotherapy 53:5235 (Salmonella enterica).

The clinical literature on actual use of Rapamycin and related drugs as immunosuppressants for renal transplant patients appears to conclusively correlate Rapamycin administration with increased incidence of Klebsiella pneumoniae infection, and probable etiology. However, this is not dispositive of the use of Rapamycin (or other mTOR inhibitors), administered appropriately for the purpose of treating these same infections.

One promising approach to combating infection is to understand host-pathogen interactions at the cellular and molecular levels in order to identify cellular pathways important for infection as well as pathogen determinants involved in disease progression. Thus, those bacterial virulence determinants implicated in modulating host-pathogen interaction are targets to design therapies based on affecting the host-pathogen interface. Conversely, identification of host cell determinants essential for bacterial infections, but transiently dispensable for the host, represent therapeutic targets in difficult-to-treat infections. Some drugs already approved for use in humans, but for purposes unrelated to antimicrobial activity, may effectively modulate the therapeutic target in the context of host-pathogen interactions.

To identify the host factors involved in suppression of host defense responses by Klebsiella pneumoniae, high-throughput screening (HTS) was performed. Specifically, the cytokine-dependent host-cells responses in presence of Klebsiella pneumoniae was analyzed in HTS after siRNA mediated knockdown of host factors using as cellular read-out nuclear translocation of NF-κB. A library of 646 kinases from a human kinome library was interrogated using as positive agonists IL-1 and TNFα, two essential cytokines in the communication of immune cells. After hit validation, a total of 22 targets showed effects above background (based on Z-score analysis) for at least 2 siRNAs for either IL-1β (18) or TNFα (12) in the hit validation. Of these validated hits, several targets were identified that can be linked to ERK and/or PI-3K-AKT signaling pathways.

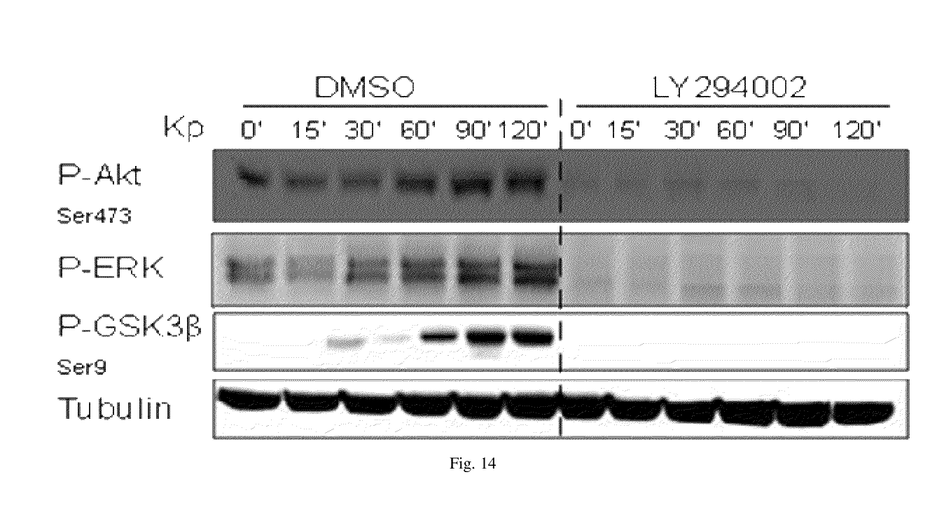

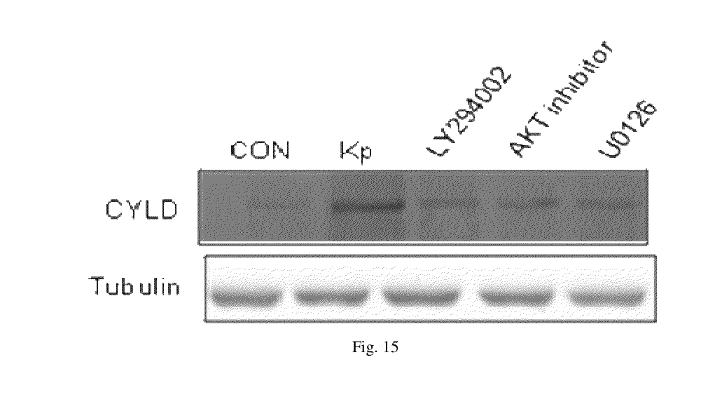

By combining bioinformatics, cell biology and immunology approaches, in-depth analysis of GSK3A, ERK, PAK4, PIK3AP1, and PIK3R1 was conducted. The data revealed that Klebsiella pneumoniae activates a pathway formed by PI3K-AKT-PAK4-ERK-mTORC1-GSK3, as shown in FIG. 14, to induce the expression of CYLD, as shown in FIG. 15, to block the activation of NF-κB induced by cytokines (either IL-1β or TNFα) and hence the production of inflammatory defensive responses.

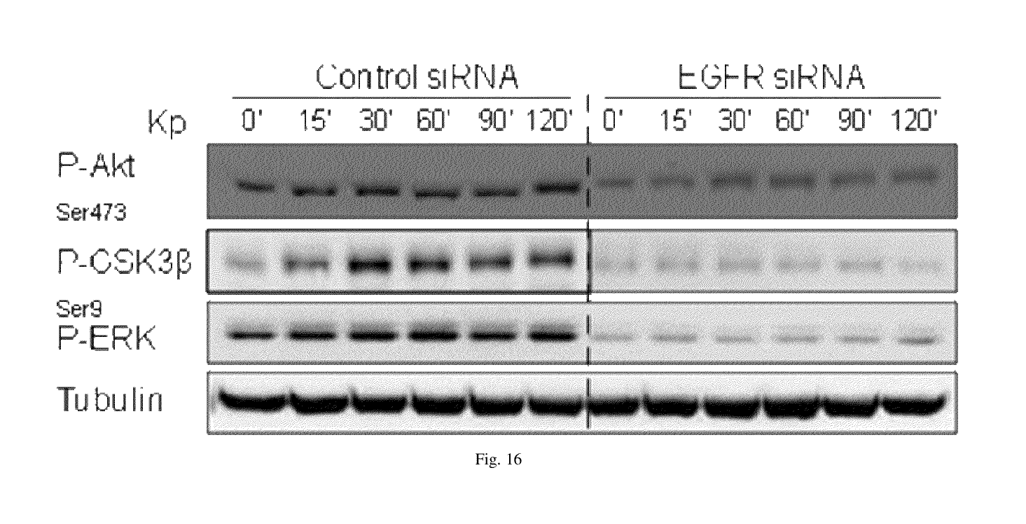

Cellular receptor(s) engaged by Klebsiella pneumoniae to exert its anti-inflammatory effect were identified by using siRNA to knock down the expression of various receptors. The potential contribution of TLR-dependent pathways to Klebsiella pneumoniae-induced anti-inflammation was investigated. However, experiments showed that there is no role for TLR signaling in Klebsiella pneumoniae anti-inflammatory effect [152]. In sharp contrast, in cells in which the intracellular receptor NOD1, belonging to the nucleotide binding and oligomerization domain-like receptors, was knocked-down, Klebsiella pneumoniae no longer elicited its anti-inflammatory effect [152]. In agreement with the previous data, in NOD 1 knockdown cells the infection-dependent phosphorylation of ERK and CYLD up-regulation was reduced. This indicated that there might be another receptor implicated in the anti-inflammatory effect. Given the connection between EGF receptor (EGFR) and PI-3K and ERK activations, the activation of EGFR by Klebsiella pneumoniae to exert its anti-inflammatory effect was studied. Further, in EGFR knockdown cells, infection-triggered PI3K-AKT-PAK4-ERK-mTORC1-GSK3 activation was attenuated, as shown in FIG. 16, Klebsiella pneumoniae-induced CYLD expression was lower, and the cytokine-dependent NF-κB activation was no longer blocked. Klebsiella pneumoniae is believed to be the first pathogen discovered hijacking NOD1 and EGFR to block the activation of inflammatory responses.

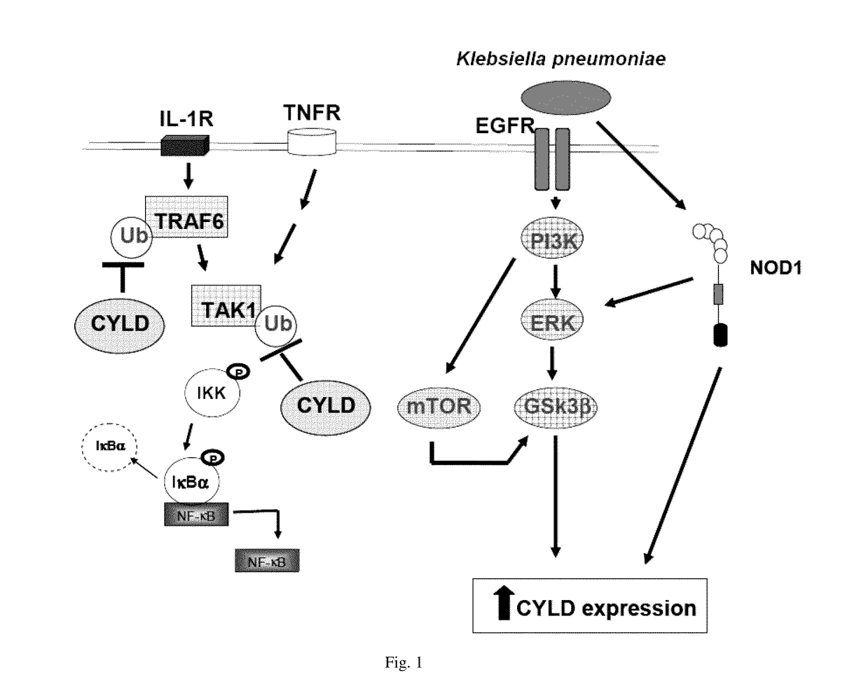

FIG. 1 shows various pathways manipulated by Klebsiella pneumoniae to block NF-κB. Klebsiella pneumoniae reduces the activation of the main cellular signaling pathways, AP-1 and NF-κB pathways, which the host turns on upon infection, to activate an inflammatory defense response. When infecting human airway epithelial cells, Klebsiella pneumoniae inhibits the cytokine-dependent nuclear translocation of NF-κB by affecting the ubiquitination status of key intermediates of the signaling pathway in a process dependent on the activation of the deubiquitinase CYLD. Klebsiella pneumoniae also targets the phosphorylation status of p38, ERK and JNK MAP kinases by activating the expression of a specific phosphatase, MKP-1. Data obtained demonstrated that Klebsiella pneumoniae induces the expression of CYLD and MKP-1 in the lungs of infected mice [152].

Human airway epithelial cells also revealed that Klebsiella pneumoniae activates mTOR in a PI3K-AKT dependent manner, as shown in FIG. 1. mTOR is the main protein controlling key cellular processes including cell growth and proliferation, cell survival and autophagy. The latter also helps to orchestrate the immune response by functioning as a regulator of innate immunity, adaptive immunity, and inflammation [153]. It has been shown that the autophagic machinery converges with the phagocytic pathway; the autolysosome is endowed with higher microbicidal potential than the phagolysosome; and therefore autophagy plays a fundamental role in eliminating intracellular bacteria [154]. Furthermore, mTOR also regulates the balance of pro and anti-inflammatory responses of macrophages. Of note, Klebsiella pneumoniae infections are characterized by an increase in anti-inflammatory mediators thereby suggesting that mTOR activity could be out of balance.

The present technology therefore addresses the interplay between Klebsiella pneumoniae and alveolar macrophages, the resident defenders of lung sterility.

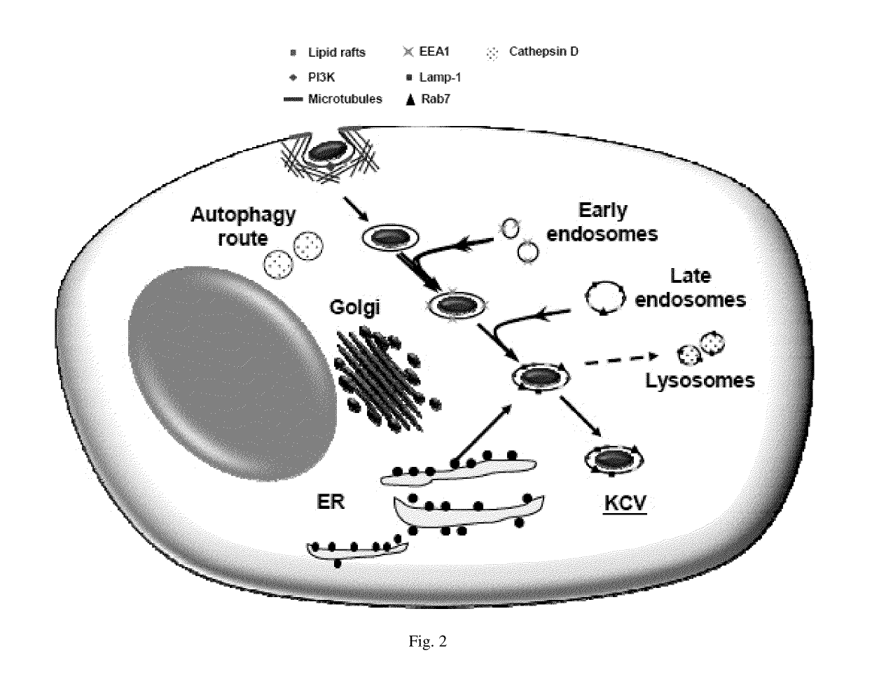

Klebsiella pneumoniae has been found to be internalized by alveolar macrophages and targeted to a phagosome-like membrane-bound organelle, the so-called Klebsiella containing vacuole (KCV), where it replicates (March, Cano and Bengoechea, unpublished results). Our data shows that Klebsiella pneumoniae precludes fusion of the KCV with lysosomes in a PI-3K-AKT dependent manner, although the means whereby Klebsiella pneumoniae co-opts phagosome maturation are currently unknown. Experimental data also confirmed that Klebsiella pneumoniae actively prevents the induction of autophagy. See FIG. 2. See, [150] [156] [157].

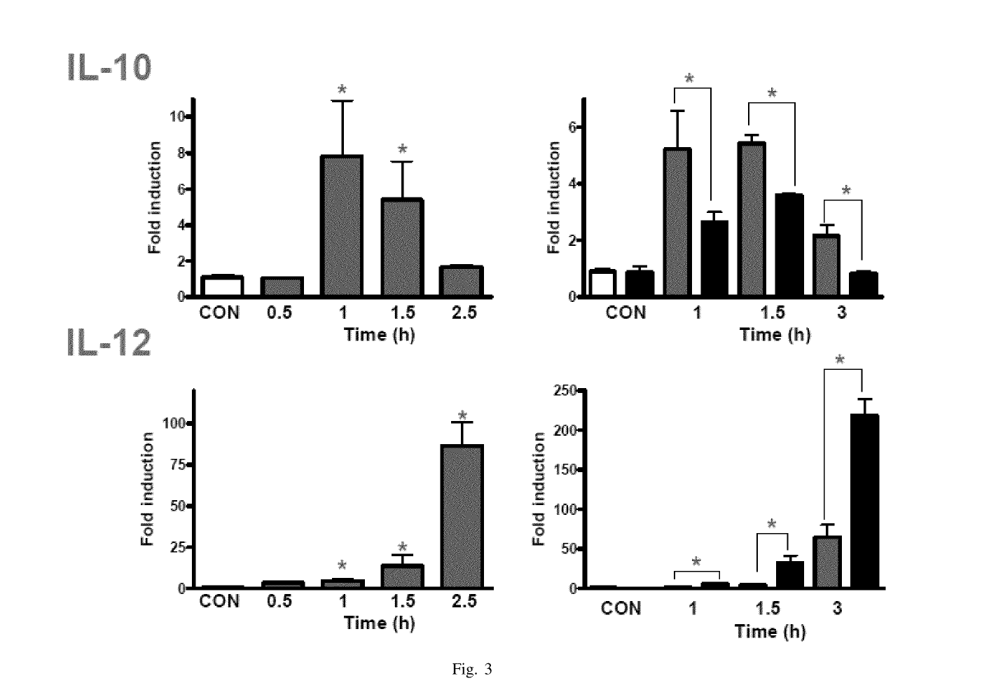

FIG. 3 shows production of IL10 (upper panels) and IL12 (lower panels) mRNA by infected macrophages for different time points. CON, non-infected cells; black bars, cells were treated with mTOR inhibitor before infection. * indicates p<0.05. In vivo studies also indicate that Klebsiella pneumoniae-triggered pneumonia is characterized by elevated levels of the anti-inflammatory cytokine IL-10 and reduced levels of IL-12 [151] [155]. Klebsiella pneumoniae may therefore instruct alveolar macrophages to engage in a specific activation program, leading to the production of anti-inflammatory cytokines. Supporting this notion, experimental data show that Klebsiella pneumoniae-infected alveolar macrophages express high levels of IL-10 and low levels of IL-12 in a process dependent on the activation of mTOR (Reguerio, Moranta and Bengoechea), shown in FIG. 3.

Collectively, this data support the notion that Klebsiella pneumoniae instructs alveolar macrophages to engage in a specific activation program leading to a favorable niche for its replication being mTOR a key cellular determinant targeted by Klebsiella pneumoniae. Therefore, by restoring the cellular function/s targeted by pathogens (Klebsiella pneumoniae in this case) the cells will clear the pathogen.

The available data demonstrates that Klebsiella pneumoniae exploits the cellular receptors NOD1, EGFR and the signaling cascade PI3K-mTOR to subvert the activation of host defense immune responses. These responses are essential to clear the infection [144] [145] [146]. Some of the molecules targeted by Klebsiella pneumoniae are also under extensive investigation as anti-cancer and anti-inflammatory drugs. For example, the prototype mTOR inhibitor, rapamycin, shows potent immunosuppressive and anti-tumor activities and it has been introduced in clinical transplantation. Whereas there are anti-EGFR drugs in used to treat breast cancer Inhibition of Klebsiella pneumoniae-hijacked factors such as mTOR and/or EGFR should help the host to clear a Klebsiella pneumoniae infection.

A pre-clinical trial was conducted to test the effect of mTOR inhibition on Klebsiella pneumoniae. Mice were prophylactically administered an mTOR inhibitor (intraperitoneal route, 1.6 mg/kg 3 h before infection), and later on infected intranasally with a highly virulent Klebsiella pneumoniae strain (mice were infected with a dose of 10⁴ colony forming units of the strain 52145; the lethal dose killing 50% of the animals of this strain is 100 bacteria). 24 h post infection, animals were sacrificed and bacterial loads in tissues determined following a published procedure (March C et al 2011 Journal Biological Chemistry 286:9956). 100 times fewer bacteria were found in the lungs of mice treated with the mTOR inhibitor than those of control treated animals. In trachea, spleen and liver, a 1-log difference between treated and control animals was observed. At the cellular level, the data demonstrated that inhibition of mTOR reduces significantly the intracellular survival of Klebsiella pneumoniae in alveolar macrophages hence providing a likely explanation of the in vivo results shown before.

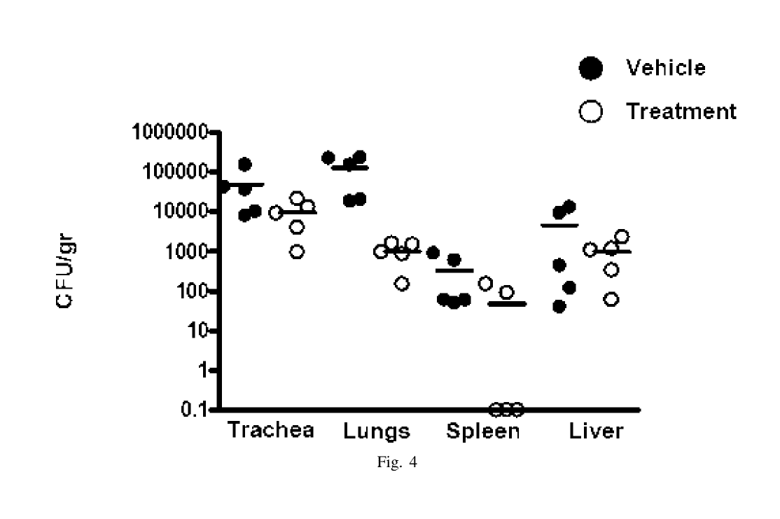

FIG. 4 shows a graph of mTOR inhibition vs. lung bacterial loads. Higher degrees of mTOR inhibition correlate with lower bacterial load.

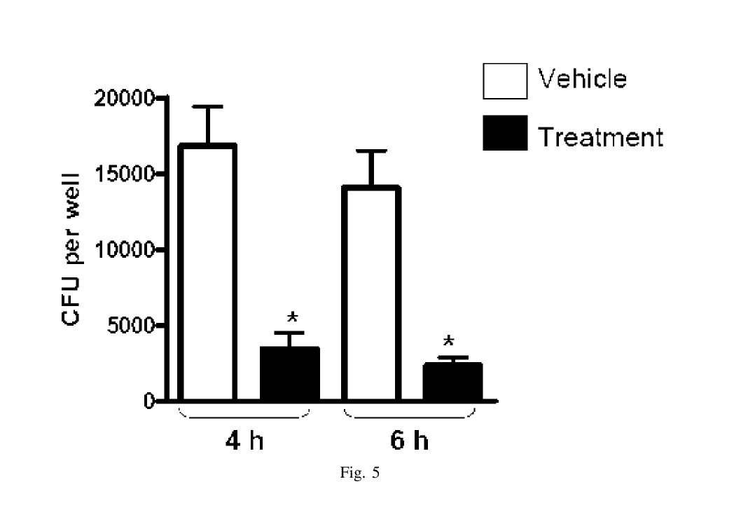

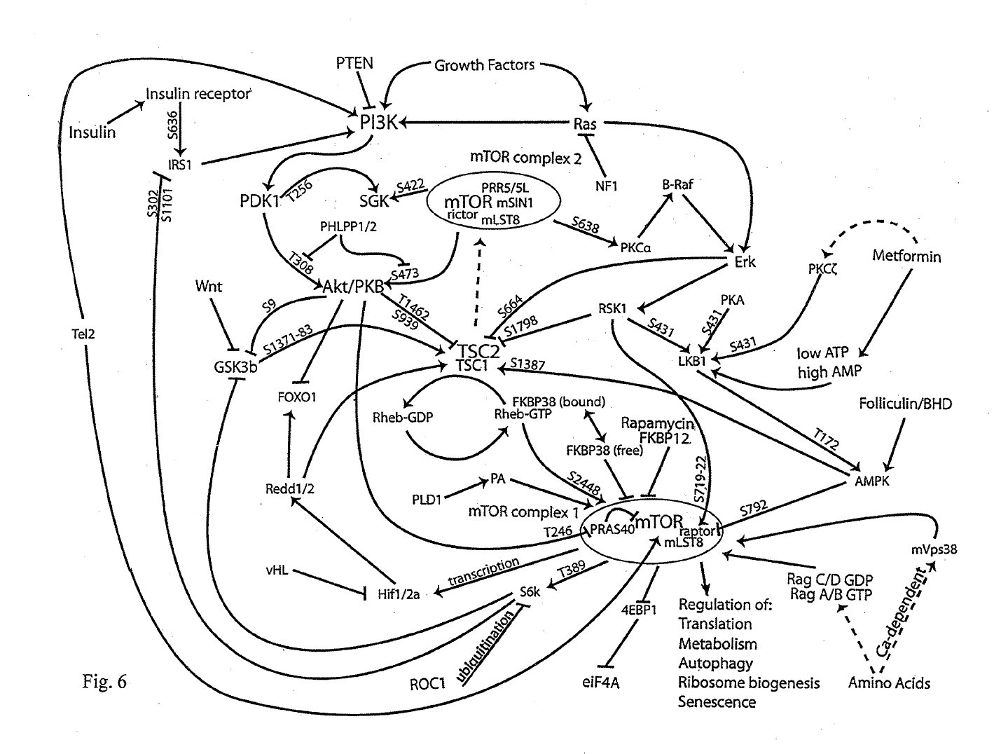

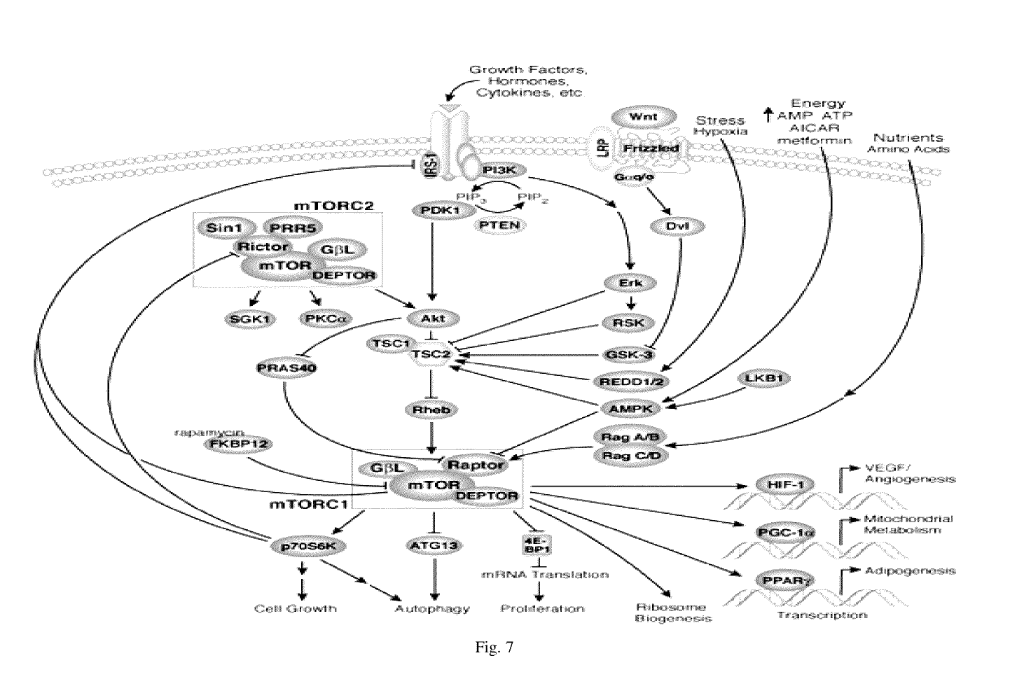

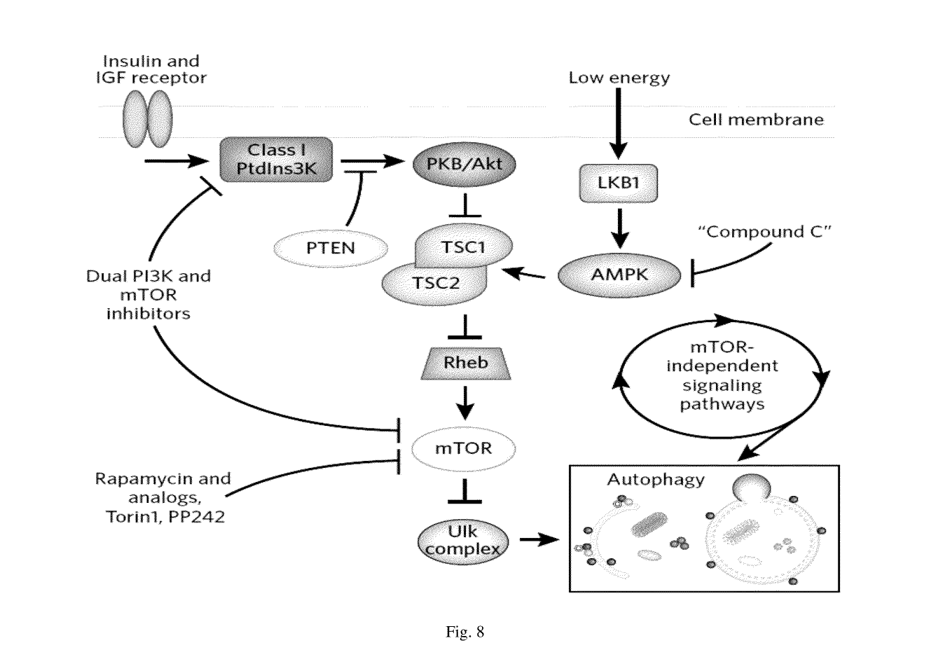

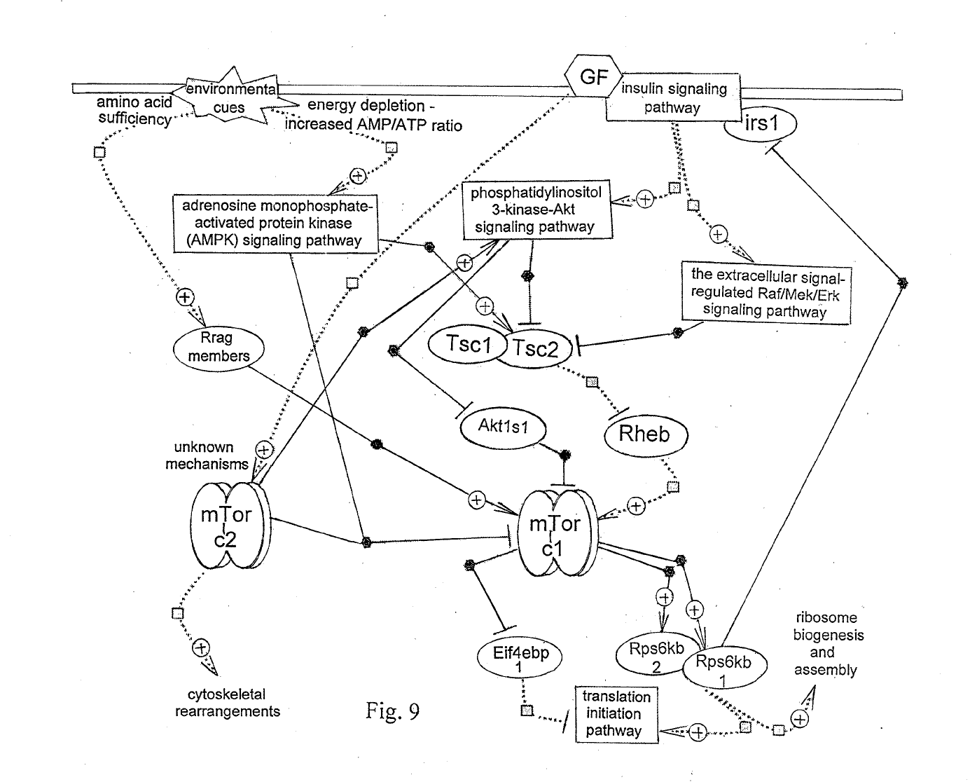

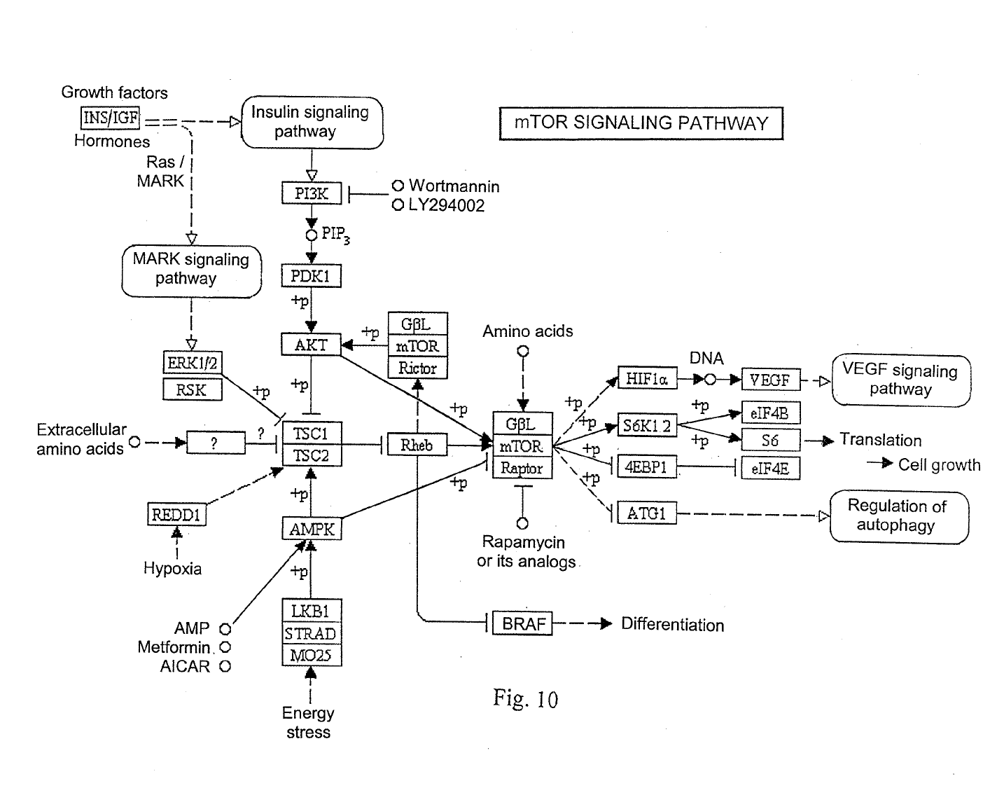

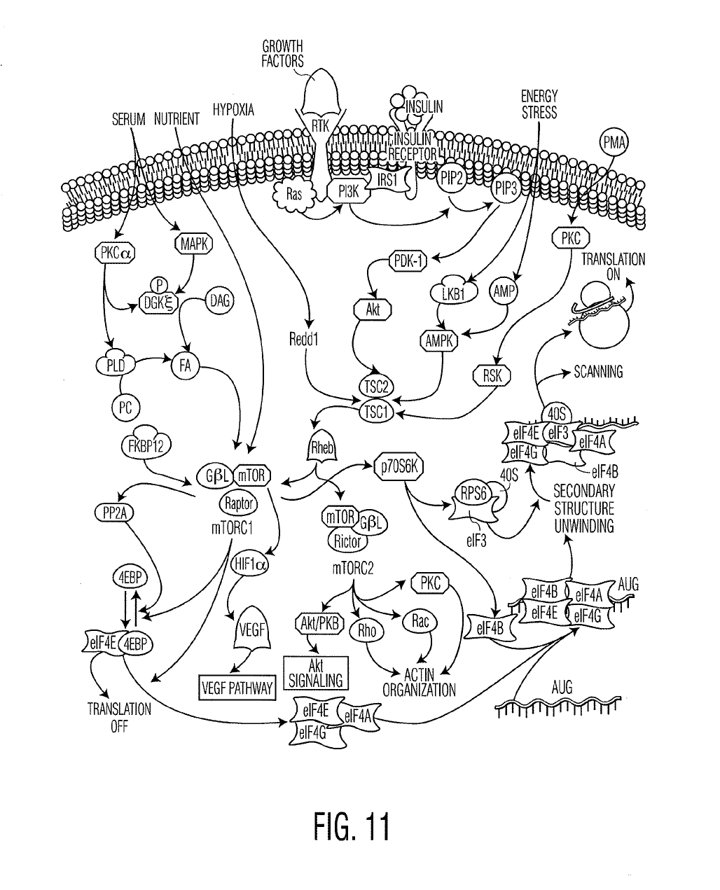

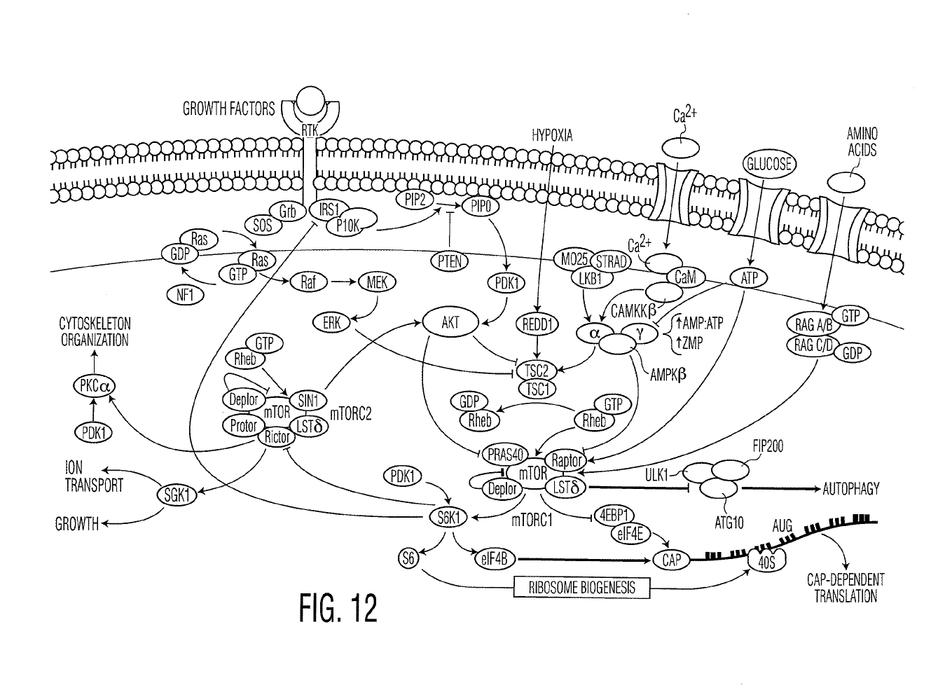

FIG. 5 shows that mTOR inhibition reduces Klebsiella pneumoniae intracellular survival. Black bars show cells that were treated with mTOR inhibitor during infection. * indicates p<0.05.

Because these inhibitors target host biology, it is less likely to engender resistance compared to conventional antibiotics, and may even decrease the development of resistance against co-administered drugs. Indeed, mTOR inhibition may also impact treatment of other respiratory infections; chiefly those triggered by the bacterial pathogens Streptococcus pneumoniae and Pseudomonas aeruginosa and the influenza and respiratory syncytial viruses. Although pathogens express different virulence determinants in all cases, they confront the same host background. Therefore, the findings with Klebsiella pneumoniae could be extrapolated to other respiratory pathogens colonizing the lung such as, but not limited to, Streptococcus pneumoniae, Mycobacterium tuberculosis, Legionella pneumophila, Pseudomonas aeruginosa, Burkholderia cenocepacia, Staphylococcus aureus and Coxiella burnetii. Therefore, the infectious pathology targeted by the present technology is not limited to Klebsiella pneumoniae induced pneumonia. Since the target of the technology is the host cell response and not the organism per se, the technology encompasses a therapy targeted against any microorganism that exploits, or might mutate or be genetically modified to exploit, the induction of the autophagy defect discussed herein.

In addition, mTOR is not the only available target, and for example, EGFR inhibitors may also prove useful. The fact that downstream of both proteins there are a few other kinases (and associated families of chemical inhibitors available) indicates that inhibition of these kinases may also restore the host ability to clear Klebsiella pneumoniae infections. These kinases, and their inhibitors, are more specific and less prone to have off-target effects.

In order to identify a best pharmacological composition to treat an organism, the kinases downstream of mTOR, PI-3K and EGFR modulated by Klebsiella pneumoniae in airway epithelial cells and alveolar macrophages are identified, for example by specific inhibition or knockout. Since the technology is host specific, the test should use cells from the same species, and preferably the same organ, as to be treated in vivo. However, for screening purposes, a mammalian model, such as mouse, may be sufficient. The effect, for example, of chemical inhibitors or siRNAs of these targets on Klebsiella pneumoniae imposed block of inflammatory responses (NF-κB activation) is then tested. Chemical inhibitors or siRNAs of these targets on Klebsiella pneumoniae intracellular survival (macrophages), and optionally other pathogenic organisms that suppress autophagy may also be tested. For those showing a suitable effect, the effect in vivo to clear Klebsiella pneumoniae from infected mice (mouse pneumonia model) may be tested. The in vivo effect to clear a panel of Klebsiella pneumoniae multidrug resistant strains alone and in combination with broad spectrum antibiotics may then tested. Drugs suitable for human administration, which appear to have promising therapeutic effects, may then be considered for administration to humans, to provide an effective treatment of Klebsiella pneumoniae, or other infectious organism responsive to the treatment. Thus, the technology may be used to develop new drugs, based on a pharmacological screening procedure according to the present technology.

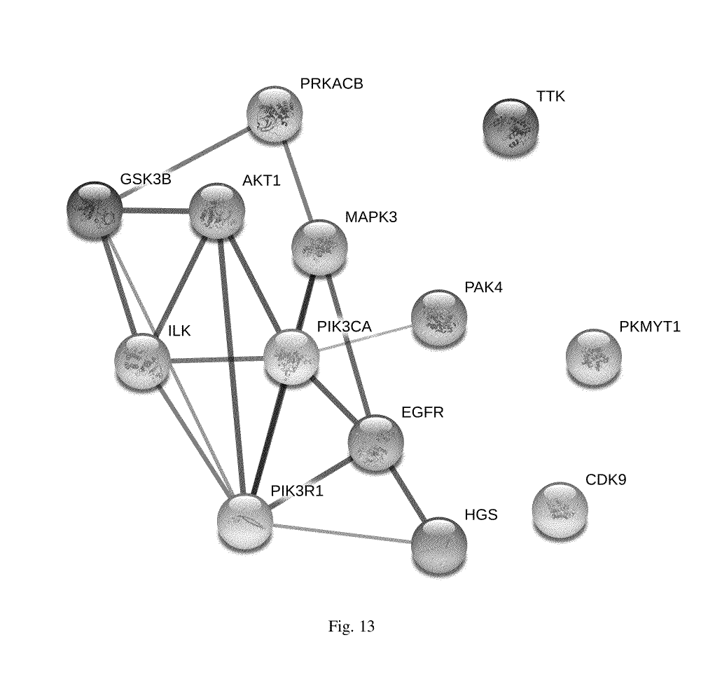

FIG. 13 shows a network of interactions predicted from text and database mining using the STRING 9.0 database. The network was seeded with the 6 high-confidence hits and components of PI3K, EGFR and ERK pathways. Thickness of connecting lines is indicative of the relative confidence score (thicker=higher; confidence value cutoff 0.5).

FIG. 14 shows that Klebsiella pneumoniae induces the phosphorylation of AKT, ERK and GSK3b in a PI3K-dependent manner. The immunoblot of the indicated proteins in cells infected with Klebsiella pneumoniae for the indicated time points in the presence of the PI3K inhibitor LY294002 (20 μM) or vehicle control DMSO. Membranes were reprobed for tubulin as a loading control.

FIG. 15 shows that Klebsiella pneumoniae induces the production of CYLD in a PI3K-AKT-ERK dependent manner. An immunoblot analysis of CYLD levels in Klebsiella pneumoniae infected cells for 3 h in the presence of vehicle control (Kp) or PI3K inhibitor (LY294002, 20 mM), AKT inhibitor (30 mM), ERK inhibitor (U0126, 10 mM) is shown. Membranes were reprobed for tubulin as a loading control.

FIG. 16 shows that Klebsiella pneumoniae induces the phosphorylation of AKT, ERK and GSK3b in an EGFR-dependent manner. The figures shows an immunoblot of the indicated proteins in cells infected with Klebsiella pneumoniae for the indicated time points in cells treated with control siRNA or knockdown for EGFR. Membranes were reprobed for tubulin as a loading control.

The dosage requirements of the rapamycin analogues can vary depending on the condition, severity of the symptoms presented and the particular subject being treated. One of skill in the art would readily be able to determine the amount of the rapamycin analogue required. In one embodiment, about 0.5 to 1000 mg is administered. In a further embodiment, about 0.5 to 250 mg is administered. In another embodiment, about 0.5 to about 100 mg is administered. In yet a further embodiment, about 1 to about 25 mg is administered. In another embodiment, about 0.5 to about 10 mg is administered, particularly when used in combination with another agent. In yet a further embodiment, about 2 to about 5 mg is administered. In yet another embodiment, about 5 to about 15 mg is administered. In general, the compositions of this invention are most desirably administered at a concentration that will generally afford effective results without causing persistent or unacceptable side effects.

In one aspect, methods of preparing a pharmaceutical composition containing one or more rapamycin analogues or mTOR inhibitors, or mTOR effector pathway inhibitors are provided, hereinafter referred to as the active composition. The active composition can be administered to a mammalian subject by several different routes and is desirably administered orally in solid or liquid form.

Rapamycin or a rapamycin analog can be obtained from any source known to those of ordinary skill in the art. The source may be a commercial source, or natural source. Rapamycin or a rapamycin analog may be chemically synthesized using any technique known to those of ordinary skill in the art. Non-Rapamycin analogs may also be used according to the present technology.

Solid forms, including tablets, capsules, and caplets, containing the rapamycin analogue can be formed by blending the active composition with one or more of the components described above. In one embodiment, the components of the active composition are dry or wet blended. In another embodiment, the components are dry granulated. In a further embodiment, the components are suspended or dissolved in a liquid and added to a form suitable for administration to a mammalian subject. Liquid forms containing the active composition can be formed by dissolving or suspending the active composition in a liquid suitable for administration to a mammalian subject. Compositions containing the active composition can be prepared according to the present invention by combining the rapamycin analogue and a pharmaceutically acceptable carrier.

The active composition can be formulated in any form suitable for the desired route of delivery using a pharmaceutically effective amount of the active composition. For example, the compositions of the invention can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release.

An oral dosage tablet composition can also be used to make oral dosage tablets containing derivatives of the active composition, including, but not limited to, esters, carbamates, sulfates, ethers, oximes, carbonates, and the like which are known to those of skill in the art.

A pharmaceutically effective amount of the active composition can vary depending on the specific compound(s), mode of delivery, severity of the condition being treated, and any other active ingredients used in the active composition. The dosing regimen can also be adjusted to provide the optimal therapeutic response. Several divided doses can be delivered daily, e.g., in divided doses 2 to 4 times a day, or a single dose can be delivered. The dose can however be proportionally reduced or increased as indicated by the exigencies of the therapeutic situation. In one embodiment, the delivery is on a daily, weekly, or monthly basis. In another embodiment, the delivery is on a daily delivery. However, daily dosages can be lowered or raised based on the periodic delivery.

The active composition can be combined with one or more pharmaceutically acceptable carriers or excipients including, without limitation, solid and liquid carriers, which are compatible with the compositions of the present invention. Such carriers include adjuvants, syrups, elixirs, diluents, binders, lubricants, surfactants, granulating agents, disintegrating agents, emollients, metal chelators, pH adjustors, surfactants, fillers, disintegrants, and combinations thereof, among others. In one embodiment, the active composition is combined with metal chelators, pH adjustors, surfactants, fillers, disintegrants, lubricants, and binders. Adjuvants can include, without limitation, flavoring agents, coloring agents, preservatives, and supplemental antioxidants, which can include vitamin E, ascorbic acid, butylated hydroxytoluene (BHT) and butylated hydroxyanisole (BHA). Binders can include, without limitation, cellulose, methylcellulose, hydroxymethylcellulose, carboxymethylcellulose calcium, carboxymethylcellulose sodium, hydroxypropylcellulose, hydroxypropylmethylcellulose phthalate, microcrystalline cellulose, noncrystalline cellulose, polypropylpyrrolidone, polyvinylpyrrolidone (povidone, PVP), gelatin, gum arabic and acacia, polyethylene glycols, starch, sugars such as sucrose, kaolin, dextrose, and lactose, cholesterol, tragacanth, stearic acid, gelatin, casein, lecithin (phosphatides), cetostearyl alcohol, cetyl alcohol, cetyl esters wax, dextrates, dextrin, glyceryl monooleate, glyceryl monostearate, glyceryl palmitostearate, polyoxyethylene alkyl ethers, polyoxyethylene castor oil derivatives, polyoxyethylene stearates, polyvinyl alcohol, and gelatin, among others. In one embodiment, the binder is povidone, hydroxypropylmethylcellulose, carboxymethylcellulose, or gelatin. In another embodiment, the binder is povidone. Lubricants can include magnesium stearate, light anhydrous silicic acid, talc, stearic acid, sodium lauryl sulfate, and sodium stearyl furamate, among others. In one embodiment, the lubricant is magnesium stearate, stearic acid, or sodium stearyl furamate. In another embodiment, the lubricant is magnesium stearate. Granulating agents can include, without limitation, silicon dioxide, microcrystalline cellulose, starch, calcium carbonate, pectin, crospovidone, and polyplasdone, among others. Disintegrating agents or disintegrants can include croscarmellose sodium, starch, carboxymethylcellulose, substituted hydroxypropylcellulose, sodium bicarbonate, calcium phosphate, calcium citrate, sodium starch glycolate, pregelatinized starch or crospovidone, among others. In one embodiment, the disintegrant is croscarmellose sodium. Emollients can include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate. Surfactants can include polysorbates, sorbitan esters, poloxamer, or sodium lauryl sulfate. In one embodiment, the surfactant is sodium lauryl sulfate. Metal chelators can include physiologically acceptable chelating agents including edetic acid, malic acid, or fumaric acid. In one embodiment, the metal chelator is edetic acid. pH adjusters can also be utilized to adjust the pH of a solution containing the rapamycin analogue to about 4 to about 6. In one embodiment, the pH of a solution containing the active composition is adjusted to a pH of about 4.6. pH adjustors can include physiologically acceptable agents including citric acid, ascorbic acid, fumaric acid, or malic acid, and salts thereof. In one embodiment, the pH adjuster is citric acid. Fillers that can be used include anhydrous lactose, microcrystalline cellulose, mannitol, calcium phosphate, pregelatinized starch, or sucrose. In one embodiment, the filler is anhydrous lactose. In another embodiment, the filler is microcrystalline cellulose.

In one embodiment, compositions containing the active composition are delivered orally by tablet, caplet or capsule, microcapsules, dispersible powder, granule, suspension, syrup, elixir, and aerosol. Desirably, when compositions containing the active composition are delivered orally, delivery is by tablets and hard- or liquid-filled capsules.

In another embodiment, the compositions containing the active composition can be delivered intravenously, intramuscularly, subcutaneously, parenterally and intraperitoneally in the form of sterile injectable solutions, suspensions, dispersions, and powders which are fluid to the extent that easy syringe ability exits. Such injectable compositions are sterile and stable under conditions of manufacture and storage, and free of the contaminating action of microorganisms such as bacteria and fungi.

In a further embodiment, compositions containing the active composition can be delivered rectally in the form of a conventional suppository.

In another embodiment, compositions containing the active composition can be delivered vaginally in the form of a conventional suppository, cream, gel, ring, or coated intrauterine device (IUD).

In another embodiment, compositions containing the active composition can be delivered via coating or impregnating of a supporting structure, i.e., a framework capable of containing of supporting pharmaceutically acceptable carrier or excipient containing an active composition, e.g., vascular stents or shunts, coronary stents, peripheral stents, catheters, arterio-venous grafts, by-pass grafts, and drug delivery balloons for use in the vasculature. In one embodiment, coatings suitable for use include, but are not limited to, polymeric coatings composed of any polymeric material in which the compound of the invention is substantially soluble. Supporting structures and coating or impregnating methods, e.g., those described in U.S. Pat. No. 6,890,546, are known to those of skill in the art.

In yet another embodiment, compositions containing the active composition can be delivered intranasally or intrabronchially in the form of an aerosol.

The active composition is administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions are advantageously included, such as flavoring agents, coloring agents, preserving agents, and antioxidants, for example, vitamin E, ascorbic acid, BHT and BHA.

The active composition is also administered parenterally or intraperitoneally. Solutions or suspensions of these active compounds as a free base or pharmacologically acceptable salt are prepared in water suitably mixed with a surfactant such as hydroxypropylcellulose. Dispersions are also prepared in glycerol, liquid, polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringe ability exits. It is stable under conditions of manufacture and storage and is preserved against the contaminating action of microorganisms such as bacterial and fungi. The carrier is a solvent or dispersion medium containing, for example, water, ethanol (e.g., glycerol, propylene glycol and liquid polyethylene glycol), suitable mixtures thereof, and vegetable oil.

The present invention also provides kits or packages containing the active composition. Kits can include the active composition and a carrier suitable for administration to a mammalian subject as discussed above.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

The entire disclosure of each document cited (including patents, patent applications, patent publications, journal articles, abstracts, laboratory manuals, books, or other disclosures) as well as information available through Identifiers specific to databases, referred to in this application are herein incorporated by reference in their entirety.

It will be understood that various details of the presently disclosed subject matter can be changed without departing from the scope of the subject matter disclosed herein. Furthermore, the foregoing description is for the purpose of illustration only, and not for the purpose of limitation.

### REFERENCE LIST

### Each of Which is Expressly Incorporated Herein by Reference, in its Entirety

1. Brown E J, Albers M W, Shin T B, Ichikawa K, Keith C T, Lane W S, Schreiber S L (June 1994). “A mammalian protein targeted by G1-arresting rapamycin-receptor complex”. Nature 369 (6483): 756-8. DOI:10.1038/369756a0. PMID 8008069.

2. Moore P A, Rosen C A, Carter K C (April 1996). “Assignment of the human FKBP12-rapamycin-associated protein (FRAP) gene to chromosome 1p36 by fluorescence in situ hybridization”. Genomics 33 (2): 331-2. DOI:10.1006/geno.1996.0206. PMID 8660990.

3. Hay N, Sonenberg N (2004). “Upstream and downstream of mTOR”. Genes Dev 18 (16): 1926-45. DOI:10.1101/gad.1212704. PMID 15314020. www.genesdev.org/cgi/content/full/18/16/1926.

4. Beevers C, Li F, Liu L, Huang S (2006). “Curcumin inhibits the mammalian target of rapamycin-mediated signaling pathways in cancer cells”. Int J Cancer 119 (4): 757-64. DOI:10.1002/ijc.21932. PMID 16550606.

5. Tokunaga C, Yoshino K, Yonezawa K (2004). “mTOR integrates amino acid- and energy-sensing pathways”. Biochem Biophys Res Commun 313 (2): 443-6. DOI:10.1016/j.bbrc.2003.07.019. PMID 14684182.

6. Huang S, Houghton P (2001). “Mechanisms of resistance to rapamycins”. Drug Resist Updat 4 (6): 378-91. DOI:10.1054/drup.2002.0227. PMID 12030785.

7. Huang S, Bjornsti M, Houghton P (2003). “Rapamycins: mechanism of action and cellular resistance”. Cancer Biol Ther 2 (3): 222-32. PMID 12878853.

8. Wullschleger S, Loewith R, Hall M (2006). “TOR signaling in growth and metabolism”. Cell 124 (3): 471-84. DOI:10.1016/j.cell.2006.01.016. PMID 16469695.

9. Kim D, Sarbassov D, Ali S, King J, Latek R, Erdjument-Bromage H, Tempst P, Sabatini D (2002). “mTOR interacts with raptor to form a nutrient-sensitive complex that signals to the cell growth machinery”. Cell 110 (2): 163-75. DOI:10.1016/S0092-8674(02)00808-5. PMID 12150925.

10. Kim D, Sarbassov D, Ali S, Latek R, Guntur K, Erdjument-Bromage H, Tempst P, Sabatini D (2003). “GbetaL, a positive regulator of the rapamycin-sensitive pathway required for the nutrient-sensitive interaction between raptor and mTOR”. Mol Cell 11 (4): 895-904. DOI:10.1016/S1097-2765(03)00114-X. PMID 12718876.

11. Fang Y, Vilella-Bach M, Bachmann R, Flanigan A, Chen J (2001). “Phosphatidic acid-mediated mitogenic activation of mTOR signaling”. Science 294 (5548): 1942-5. DOI:10.1126/science.1066015. PMID 11729323.

12. Sancak Y, Peterson T R, Shaul Y D, Lindquist R A, Thoreen C C, Bar-Peled L, Sabatini D M (June 2008). “The Rag GTPases bind raptor and mediate amino acid signaling to mTORC1”. Science 320 (5882): 1496-501. DOI:10.1126/science.1157535. PMC 2475333. PMID 18497260. www.pubmedcentral.nih.gov/articlerender.fcgi?tool=pmcentrez&artid=2475333.

13. Saucedo L J, Gao X, Chiarelli D A, Li L, Pan D, Edgar B A (June 2003). “Rheb promotes cell growth as a component of the insulin/TOR signalling network”. Nat. Cell Biol. 5 (6): 566-71. DOI:10.1038/ncb996. PMID 12766776.

14. Zoncu R, Efeyan A, Sabatini D M (January 2011). “mTOR: from growth signal integration to cancer, diabetes and ageing”. Nat. Rev. Mol. Cell Biol. 12 (1): 21-35. DOI:10.1038/nrm3025. PMID 21157483.

15. Inoki K, Li Y, Zhu T, Wu J, Guan K L (September 2002). “TSC2 is phosphorylated and inhibited by Akt and suppresses mTOR signalling”. Nat. Cell Biol. 4 (9): 648-57. DOI:10.1038/ncb839. PMID 12172553.

16. Ma L, Chen Z, Erdjument-Bromage H, Tempst P, Pandolfi P P (April 2005). “Phosphorylation and functional inactivation of TSC2 by Erk implications for tuberous sclerosis and cancer pathogenesis”. Cell 121 (2): 179-93. DOI:10.1016/j.cell.2005.02.031. PMID 15851026.

17. Hardie D G (October 2007). “AMP-activated/SNF1 protein kinases: conserved guardians of cellular energy”. Nat. Rev. Mol. Cell Biol. 8 (10): 774-85. DOI:10.1038/nrm2249. PMID 17712357.

18. Brugarolas J, Lei K, Hurley R L, Manning B D, Reiling J H, Hafen E, Witters L A, Ellisen L W, Kaelin W G (December 2004). “Regulation of mTOR function in response to hypoxia by REDD1 and the TSC1/TSC2 tumor suppressor complex”. Genes Dev. 18 (23): 2893-904. DOI:10.1101/gad.1256804. PMC 534650. PMID 15545625. www.pubmedcentral.nih.gov/articlerender.fcgi?tool=pmcentrez&artid=534650.

19. Saitoh M, Pullen N, Brennan P, Cantrell D, Dennis P B, Thomas G (May 2002). “Regulation of an activated S6 kinase 1 variant reveals a novel mammalian target of rapamycin phosphorylation site”. J. Biol. Chem. 277 (22): 20104-12. DOI:10.1074/jbc.M201745200. PMID 11914378.

20. Pullen N, Thomas G (June 1997). “The modular phosphorylation and activation of p70s6k”. FEBS Lett. 410 (1): 78-82. DOI:10.1016/S0014-5793(97)00323-2. PMID 9247127.

21. Pullen N, Dennis P B, Andjelkovic M, Dufner A, Kozma S C, Hemmings B A, Thomas G (January 1998). “Phosphorylation and activation of p70s6k by PDK1”. Science 279 (5351): 707-10. DOI:10.1126/science.279.5351.707. PMID 9445476.

22. Peterson R T, Schreiber S L (March 1998). “Translation control: connecting mitogens and the ribosome”. Curr. Biol. 8 (7): R248-50. DOI:10.1016/50960-9822(98)70152-6. PMID 9545190.

23. Chiang G G, Abraham R T (July 2005). “Phosphorylation of mammalian target of rapamycin (mTOR) at Ser-2448 is mediated by p70S6 kinase”. J. Biol. Chem. 280 (27): 25485-90. DOI:10.1074/jbc.M501707200. PMID 15899889.

24. Holz M K, Blenis J (July 2005). “Identification of S6 kinase 1 as a novel mammalian target of rapamycin (mTOR)-phosphorylating kinase”. J. Biol. Chem. 280 (28): 26089-93. DOI:10.1074/jbc.M504045200. PMID 15905173.

25. Gingras A C, Gygi S P, Raught B, Polakiewicz R D, Abraham R T, Hoekstra M F, Aebersold R, Sonenberg N (June 1999). “Regulation of 4E-BP1 phosphorylation: a novel two-step mechanism”. Genes Dev. 13 (11): 1422-37. DOI:10.1101/gad.13.11.1422. PMC 316780. PMID 10364159. www.pubmedcentral.nih gov/articlerender.fcgi?tool=pmcentrez&artid=316780.

26. Mothe-Satney I, Brunn G J, McMahon L P, Capaldo C T, Abraham R T, Lawrence J C (October 2000). “Mammalian target of rapamycin-dependent phosphorylation of PHAS-I in four (S/T)P sites detected by phospho-specific antibodies”. J. Biol. Chem. 275 (43): 33836-43. DOI:10.1074/jbc.M006005200. PMID 10942774.

27. Pause A, Belsham G J, Gingras A C, Donzé O, Lin T A, Lawrence J C, Sonenberg N (October 1994). “Insulin-dependent stimulation of protein synthesis by phosphorylation of a regulator of 5′-cap function”. Nature 371 (6500): 762-7. DOI: 10.1038/371762a0. PMID 7935836.

28. Frias M, Thoreen C, Jaffe J, Schroder W, Sculley T, Carr S, Sabatini D (2006). “mSin1 is necessary for Akt/PKB phosphorylation, and its isoforms define three distinct mTORC2s”. Curr Biol 16 (18): 1865-70. DOI:10.1016/j.cub.2006.08.001. PMID 16919458.

29. Sarbassov D, Ali S, Kim D, Guertin D, Latek R, Erdjument-Bromage H, Tempst P, Sabatini D (2004). “Rictor, a novel binding partner of mTOR, defines a rapamycin-insensitive and raptor-independent pathway that regulates the cytoskeleton”. Curr Biol 14 (14): 1296-302. DOI:10.1016/j.cub.2004.06.054. PMID 15268862.

30. Sarbassov D, Guertin D, Ali S, Sabatini D (2005). “Phosphorylation and regulation of Akt/PKB by the rictor-mTOR complex”. Science 307 (5712): 1098-101. DOI:10.1126/science.1106148. PMID 15718470.

31. Stephens L, Anderson K, Stokoe D, Erdjument-Bromage H, Painter G, Holmes A, Gaffney P, Reese C, McCormick F, Tempst P, Coadwell J, Hawkins P (1998). “Protein kinase B kinases that mediate phosphatidylinositol 3,4,5-trisphosphate-dependent activation of protein kinase B”. Science 279 (5351): 710-4. DOI:10.1126/science.279.5351.710. PMID 9445477.

32. Sarbassov D, Ali S, Sengupta S, Sheen J, Hsu P, Bagley A, Markhard A, Sabatini D (2006). “Prolonged rapamycin treatment inhibits mTORC2 assembly and Akt/PKB”. Mol Cell 22 (2): 159-68. DOI:10.1016/j.molcel.2006.03.029. PMID 16603397.

33. Lamming D W, Ye L, Katajisto P, et al. (March 2012). “Rapamycin-induced insulin resistance is mediated by mTORC2 loss and uncoupled from longevity”. Science 335 (6076): 1638-43. DOI:10.1126/science.1215135. PMID 22461615.

34. Zinzalla V, Stracka D, Oppliger W, Hall M N (March 2011). “Activation of mTORC2 by association with the ribosome”. Cell 144 (5): 757-68. DOI:10.1016/j.cell.2011.02.014. PMID 21376236.

35. Zhang F, Zhang X, Li M, Chen P, Zhang B, Guo H, Cao W, Wei X, Cao X, Hao X, Zhang N (November 2010). “mTOR complex component Rictor interacts with PKCzeta and regulates cancer cell metastasis”. Cancer Res. 70 (22): 9360-70. DOI:10.1158/0008-5472.CAN-10-0207. PMID 20978191.

36. Feldman M E, Apsel B, Uotila A, Loewith R, Knight Z A, Ruggero D, Shokat K M (February 2009). Hunter, Tony. ed. “Active-Site Inhibitors of mTOR Target Rapamycin-Resistant Outputs of mTORC1 and mTORC2”. PLoS Biol. 7 (2): e38. DOI:10.1371/journal.pbio.1000038. PMC 2637922. PMID 19209957. www.pubmedcentral.nih gov/articlerender.fcgi?tool=pmcentrez&artid=2637922.

37. Lawlor M A, Mora A, Ashby P R, Williams M R, Murray-Tait V, Malone L, Prescott A R, Lucocq J M, Alessi D R (July 2002). “Essential role of PDK1 in regulating cell size and development in mice”. EMBO J. 21 (14): 3728-38. DOI:10.1093/emboj/cdf387. PMC 126129. PMID 12110585. www.pubmedcentral.nih gov/articlerender.fcgi?tool=pmcentrez&artid=126129.

38. Yang Z Z, Tschopp O, Baudry A, Dümmler B, Hynx D, Hemmings B A (April 2004). “Physiological functions of protein kinase B/Akt”. Biochem. Soc. Trans. 32 (Pt 2): 350-4. DOI:10.1042/BST0320350. PMID 15046607.

39. Powers R W, Kaeberlein M, Caldwell S D, Kennedy B K, Fields S (January 2006). “Extension of chronological life span in yeast by decreased TOR pathway signaling”. Genes Dev. 20 (2): 174-84. DOI:10.1101/gad.1381406. PMC 1356109. PMID 16418483. www.pubmedcentral.nih gov/articlerender.fcgi?tool=pmcentrez&artid=1356109.

40. Kaeberlein M, Powers R W, Steffen K K, Westman E A, Hu D, Dang N, Kerr E O, Kirkland K T, Fields S, Kennedy B K (November 2005). “Regulation of yeast replicative life span by TOR and Sch9 in response to nutrients”. Science 310 (5751): 1193-6. DOI:10.1126/science.1115535. PMID 16293764.

41. Jia K, Chen D, Riddle D L (August 2004). “The TOR pathway interacts with the insulin signaling pathway to regulate C. elegans larval development, metabolism and life span”. Development 131 (16): 3897-906. DOI:10.1242/dev.01255. PMID 15253933.

42. Kapahi P, Zid B M, Harper T, Koslover D, Sapin V, Benzer S (May 2004). “Regulation of Lifespan in Drosophila by Modulation of Genes in the TOR Signaling Pathway”. Curr. Biol. 14 (10): 885-90. DOI:10.1016/j.cub.2004.03.059. PMC 2754830. PMID 15186745. www.pubmedcentral.nih gov/articlerender.fcgi?tool=pmcentrez&artid=2754830.

43. Harrison D E, Strong R et al. (July 8 2009). “Rapamycin fed late in life extends lifespan in genetically heterogeneous mice”. Nature 460 (AOP): 392-5. DOI:10.1038/nature08221. PMC 2786175. PMID 19587680. www.nature.com/nature/journal/vaop/ncurrent/full/nature08221.html.

44. Cota D, Proulx K, Smith K A, Kozma S C, Thomas G, Woods S C, Seeley R J (2006). “Hypothalamic mTOR signaling regulates food intake”. Science 312 (5775): 927-930. DOI:10.1126/science.1124147. PMID 16690869.

45. Rosner M, Hanneder M, Siegel N, Valli A, Fuchs C, Hengstschlager M. (June 2008). “The mTOR pathway and its role in human genetic diseases”. Mutat. Res. 659 (3): 284-92. PMID 18598780.

46. Li X, Alafuzoff I, Soininen H, Winblad B, Pei J J. (August 2005). “Levels of mTOR and its downstream targets 4E-BP1, eEF2, and eEF2 kinase in relationships with tau in Alzheimer's disease brain”. FEBS J. 272 (16): 4211-20. PMID 16098202.

47. Chano T, Okabe H, Hulette C M. (July 2007). “RB1CC1 insufficiency causes neuronal atrophy through mTOR signaling alteration and involved in the pathology of Alzheimer's diseases”. Brain Res. (1168): 97-105. PMID 17706618.

48. Selkoe D J (September 2008). “Soluble oligomers of the amyloid beta-protein impair synaptic plasticity and behavior”. Behav Brain Res. 192 (1): 106-13. PMID 18359102.

49. Oddo S (January 2012). “The role of mTOR signaling in Alzheimer disease”. Front Biosci. (4): 941-52. PMID 22202101.

50. An W L, Cowburn R F, Li L, Braak H, Alafuzoff I, Iqbal K, Iqbal I G, Winblad B, Pei J J. (August 2003). “Up-regulation of phosphorylated/activated p70 S6 kinase and its relationship to neurofibrillary pathology in Alzheimer's disease”. Am J Pathol. 163 (2): 591-607. PMID 12875979.

51. Zhang F, Beharry Z M, Harris T E, Lilly M B, Smith C D, Mahajan S, Kraft A S. (May 2009). “PIM1 protein kinase regulates PRAS40 phosphorylation and mTOR activity in FDCP1 cells”. Cancer Biol Ther. 8 (9): 846-53. PMID 19276681.

52. Koo E H, Squazzo S L. (July 1994). “Evidence that production and release of amyloid beta-protein involves the endocytic pathway”. J Biol Chem. 269 (26): 17386-9. PMID 8021238.

53. Caccamo A, Majumder S, Richardson A, Strong R, Oddo S. (April 2010). “Molecular interplay between mammalian target of rapamycin (mTOR), amyloid-beta, and Tau: effects on cognitive impairments”. J Biol Chem. 285 (17): 13107-20. PMID 20178983.

54. Lafay-Chebassier C, Paccalin M, Page G, Barc-Pain S, Perault-Pochat M C, Gil R, Pradier L, Hugon J. (July 2005). “mTOR/p70S6k signalling alteration by Abeta exposure as well as in APP-PS 1 transgenic models and in patients with Alzheimer's disease”. J Neurochem. 94 (1): 215-25. PMID 15953364.

55. Caccamo A, Maldonado M A, Majumder S, Medina D X, Holbein W, Magrí A, Oddo S. (March 2011). “Naturally secreted amyloid-beta increases mammalian target of rapamycin (mTOR) activity via a PRAS40-mediated mechanism”. J Biol Chem. 286 (11): 8924-32. PMID 21266573.

56. Sancak Y, Thoreen C C, Peterson T R, Lindquist R A, Kang S A, Spooner E, Carr S A, Sabatini D M. (March 2007). “PRAS40 is an insulin-regulated inhibitor of the mTORC1 protein kinase”. Mol Cell. 25 (6): 903-15. PMID 17386266.

57. Wang L, Harris T E, Roth R A, Lawrence J C Jr. (July 2007). “PRAS40 regulates mTORC1 kinase activity by functioning as a direct inhibitor of substrate binding”. J. Biol Chem. 282 (27): 20036-44. PMID 17510057.

58. Pei J J, Hugon J. (December 2008). “mTOR-dependent signalling in Alzheimer's disease”. J Cell Mol Med. 12 (6B): 2525-32. PMID 19210753.

59. Meske V, Albert F, Ohm T G. (January 2008). “Coupling of mammalian target of rapamycin with phosphoinositide 3-kinase signaling pathway regulates protein phosphatase 2A- and glycogen synthase kinase-3-dependent phosphorylation of Tau”. J Biol Chem. 293 (1): 100-9. PMID 17971449.

60. Janssens V, Goris J. (February 2001). “Protein phosphatase 2A: a highly regulated family of serine/threonine phosphatases implicated in cell growth and signalling”. Biochem J. 353 (Pt 3): 417-39. PMID 11171037.

61. Morita T, Sobue K. (October 2009). “Specification of neuronal polarity regulated by local translation of CRMP2 and Tau via the mTOR-p70S6K pathway”. J Biol Chem. 284 (40): 27734-45. PMID 19648118.

62. Puighermanal E, Marsicano G, Busquets-Garcia A, Lutz B, Maldonado R, Ozaita A. (September 2009). “Cannabinoid modulation of hippocampal long-term memory is mediated by mTOR signaling”. Nat Neurosci. 12 (9): 1152-8. PMID 19648913.

63. Tischmeyer W, Schicknick H, Kraus M, Seidenbecher C I, Staak S, Scheich H, Gundelfinger E D. (August 2003). “Rapamycin-sensitive signalling in long-term consolidation of auditory cortex-dependent memory”. Eur J Neurosci. 18 (4): 942-50. PMID 12925020.

64. Hoeffer C A, Klann E. (February 2010). “mTOR signaling: at the crossroads of plasticity, memory and disease”. Trends Neurosci. 33 (2): 67-75. PMID 19963289.

65. Kelleher R J 3rd, Govindarajan A, Jung H Y, Kang H, Tonegawa S. (February 2004). “Translational control by MAPK signaling in long-term synaptic plasticity and memory”. Cell. 116 (3): 467-79. PMID 15016380.

66. Ehninger D, Han S, Shilyansky C, Zhou Y, Li W, Kwiatkowski D J, Ramesh V, Silva A J. (August 2008). “Reversal of learning deficits in a Tsc2+/− mouse model of tuberous sclerosis”. Nat Med. 14 (8): 843-8. PMID 18568033.

67. Julie A. Moreno, Helois Radford, Diego Peretti, Joern R. Steinert, Nicholas Verity, Maria Guerra Martin, Mark Halliday, Jason Morgan, David Dinsdale, Catherine A. Ortori, David A. Barrett, Pavel Tsaytler, Anne Bertolotti, Anne E. Willis, Martin Bushell & Giovanna R. Mallucci (March 2012). “Sustained translational repression by eIF2α-P mediates prion neurodegeneration”. Nature.

68. Díaz-Troya S, Perez-Perez M E, Florencio F J, Crespo J L. (October 2008). “The role of TOR in autophagy regulation from yeast to plants and mammals”. Autophagy. 4 (7): 851-65. PMID 18670193.

69. McCray B A, Taylor J P. (December 2008). “The role of autophagy in age-related neurodegeneration”. Neurosignals. 16 (1): 75-84. PMID 18097162.

70. Nedelsky N B, Todd P K, Taylor J P. (December 2008). “Autophagy and the ubiquitin-proteasome system: collaborators in neuroprotection”. Biochim Biophys Acta. 1782 (12): 691-9. PMID 18930136.

71. David C. Rubinsztein (October 2006). “The roles of intracellular protein-degradation pathways in neurodegeneration”. Nature: 780-786.

72. Oddo S. (April 2008). “The ubiquitin-proteasome system in Alzheimer's disease”. J Cell Mol Med. 12 (2): 363-73. PMID 18266959.

73. Li X, Li H, Li X J. (November 2008). “Intracellular degradation of misfolded proteins in polyglutamine neurodegenerative diseases”. Brain Res Rev. 59 (1): 245-52. PMID 18773920.

74. Caccamo A, Majumder S, Deng J J, Bai Y, Thornton F B, Oddo S. (October 2009). “Rapamycin rescues TDP-43 mislocalization and the associated low molecular mass neurofilament instability”. J Biol Chem. 284 (40): 27416-24. PMID 19651785.

75. Ravikumar B, Vacher C, Berger Z, Davies J E, Luo S, Oroz L G, Scaravilli F, Easton D F, Duden R, O'Kane C J, Rubinsztein D C. (June 2004) “Inhibition of mTOR induces autophagy and reduces toxicity of polyglutamine expansions in fly and mouse models of Huntington disease”. Nat Genet. 36 (6): 585-95. PMID 15146184.

76. Rami A. (October 2009). “Review: autophagy in neurodegeneration: firefighter and/or incendiarist?”. Neuropathol Appl Neurobio. 35 (5): 449-61. PMID 19555462.

77. Zhou H, Luo Y, Huang S (September 2010). “Updates of mTOR inhibitors”. Anticancer Agents Med Chem 10 (7): 571-81. PMC 2980558. PMID 20812900. www.pubmedcentral.nih gov/articlerender.fcgi?tool=pmcentrez&artid=2980558.

78. Easton J B, Houghton P J (October 2006). “mTOR and cancer therapy”. Oncogene 25 (48): 6436-46. DOI:10.1038/sj.onc.1209886. PMID 17041628.

79. Faivre S, Kroemer G, Raymond E (August 2006). “Current development of mTOR inhibitors as anticancer agents”. Nat Rev Drug Discov 5 (8): 671-88. DOI:10.1038/nrd2062. PMID 16883305.

80. Hasty P (February 2010). “Rapamycin: the cure for all that ails”. J Mol Cell Biol 2 (1): 17-9. DOI:10.1093/jmcb/mjp033. PMID 19805415.

81. “mTOR protein interactors”. Human Protein Reference Database. Johns Hopkins University and the Institute of Bioinformatics. www.hprd.org/interactions?hprd_id=03134&isoform_id=03134_{—}1&isoform_name=. Retrieved Dec. 6, 2010.

82. Kumar V, Sabatini D, Pandey P, Gingras A C, Majumder P K, Kumar M, Yuan Z M, Carmichael G, Weichselbaum R, Sonenberg N, Kufe D, Kharbanda S (April 2000). “Regulation of the rapamycin and FKBP-target 1/mammalian target of rapamycin and cap-dependent initiation of translation by the c-Abl protein-tyrosine kinase”. J. Biol. Chem. 275 (15): 10779-87. DOI:10.1074/jbc.275.15.10779. PMID 10753870.

83. Sarbassov D D, Guertin D A, Ali S M, Sabatini D M (February 2005). “Phosphorylation and regulation of Akt/PKB by the rictor-mTOR complex”. Science 307 (5712): 1098-101. DOI:10.1126/science.1106148. PMID 15718470.

84. Sekulié A, Hudson C C, Homme J L, Yin P, Otterness D M, Karnitz L M, Abraham R T (July 2000). “A direct linkage between the phosphoinositide 3-kinase-AKT signaling pathway and the mammalian target of rapamycin in mitogen-stimulated and transformed cells”. Cancer Res. 60 (13): 3504-13. PMID 10910062.

85. Cheng S W, Fryer L G, Carling D, Shepherd P R (April 2004). “Thr2446 is a novel mammalian target of rapamycin (mTOR) phosphorylation site regulated by nutrient status”. J. Biol. Chem. 279 (16): 15719-22. DOI:10.1074/jbc.C300534200. PMID 14970221.

86. A Choi J H, Bertram P G, Drenan R, Carvalho J, Zhou H H, Zheng X F (October 2002). “The FKBP12-rapamycin-associated protein (FRAP) is a CLIP-170 kinase”. EMBO Rep. 3 (10): 988-94. DOI:10.1093/embo-reports/kvf197. PMC 1307618. PMID 12231510. www.pubmedcentral.nih gov/articlerender.fcgi?tool=pmcentrez&artid=1307618.

87. Harris T E, Chi A, Shabanowitz J, Hunt D F, Rhoads R E, Lawrence J C (April 2006). “mTOR-dependent stimulation of the association of eIF4G and eIF3 by insulin”. EMBO J. 25 (8): 1659-68. DOI:10.1038/sj.emboj.7601047. PMC 1440840. PMID 16541103. www.pubmedcentral.nih gov/articlerender.fcgi?tool=pmcentrez&artid=1440840.

88. Schalm S S, Fingar D C, Sabatini D M, Blenis J (May 2003). “TOS motif-mediated raptor binding regulates 4E-BP1 multisite phosphorylation and function”. Curr. Biol. 13 (10): 797-806. DOI:10.1016/S0960-9822(03)00329-4. PMID 12747827.

89. Hara K, Maruki Y, Long X, Yoshino K, Oshiro N, Hidayat S, Tokunaga C, Avruch J, Yonezawa K (July 2002). “Raptor, a binding partner of target of rapamycin (TOR), mediates TOR action”. Cell 110 (2): 177-89. DOI:10.1016/S0092-8674(02)00833-4. PMID 12150926.

90. Wang L, Rhodes C J, Lawrence J C (August 2006). “Activation of mammalian target of rapamycin (mTOR) by insulin is associated with stimulation of 4EBP1 binding to dimeric mTOR complex 1”. J. Biol. Chem. 281 (34): 24293-303. DOI:10.1074/jbc.M603566200. PMID 16798736.

91. Kim D H, Sarbassov D D, Ali S M, King J E, Latek R R, Erdjument-Bromage H, Tempst P, Sabatini D M (July 2002). “mTOR interacts with raptor to form a nutrient-sensitive complex that signals to the cell growth machinery”. Cell 110 (2): 163-75. DOI:10.1016/S0092-8674(02)00808-5. PMID 12150925.

92. Long X, Lin Y, Ortiz-Vega S, Yonezawa K, Avruch J (April 2005). “Rheb binds and regulates the mTOR kinase”. Curr. Biol. 15 (8): 702-13. DOI:10.1016/j.cub.2005.02.053. PMID 15854902.

93. Takahashi T, Hara K, Inoue H, Kawa Y, Tokunaga C, Hidayat S, Yoshino K, Kuroda Y, Yonezawa K (September 2000). “Carboxyl-terminal region conserved among phosphoinositide-kinase-related kinases is indispensable for mTOR function in vivo and in vitro”. Genes Cells 5 (9): 765-75. DOI:10.1073/pnas.95.4.1432. PMID 10971657.

94. Burnett P E, Barrow R K, Cohen N A, Snyder S H, Sabatini D M (February 1998). “RAFT1 phosphorylation of the translational regulators p70 S6 kinase and 4E-BP1”. Proc. Natl. Acad. Sci. U.S.A. 95 (4): 1432-7. DOI:10.1073/pnas.95.4.1432. PMC 19032. PMID 9465032. www.pubmedcentral.nih gov/articlerender.fcgi?tool=pmcentrez&artid=19032.

95. Wang X, Beugnet A, Murakami M, Yamanaka S, Proud C G (April 2005). “Distinct signaling events downstream of mTOR cooperate to mediate the effects of amino acids and insulin on initiation factor 4E-binding proteins”. Mol. Cell. Biol. 25 (7): 2558-72. DOI:10.1128/MCB.25.7.2558-2572.2005. PMC 1061630. PMID 15767663. www.pubmedcentral.nih gov/articlerender.fcgi?tool=pmcentrez&artid=1061630.

96. Jacinto E, Loewith R, Schmidt A, Lin S, Rüegg M A, Hall A, Hall M N (November 2004). “Mammalian TOR complex 2 controls the actin cytoskeleton and is rapamycin insensitive”. Nat. Cell Biol. 6 (11): 1122-8. DOI:10.1038/ncb1183. PMID 15467718.

97. Sarbassov D D, Ali S M, Kim D H, Guertin D A, Latek R R, Erdjument-Bromage H, Tempst P, Sabatini D M (July 2004). “Rictor, a novel binding partner of mTOR, defines a rapamycin-insensitive and raptor-independent pathway that regulates the cytoskeleton”. Curr. Biol. 14 (14): 1296-302. DOI:10.1016/j.cub.2004.06.054. PMID 15268862.

98. Choi J, Chen J, Schreiber S L, Clardy J (July 1996). “Structure of the FKBP12-rapamycin complex interacting with the binding domain of human FRAP”. Science 273 (5272): 239-42. DOI:10.1126/science.273.5272.239. PMID 8662507.

99. Luker K E, Smith M C, Luker G D, Gammon S T, Piwnica-Worms H, Piwnica-Worms D (August 2004). “Kinetics of regulated protein-protein interactions revealed with firefly luciferase complementation imaging in cells and living animals”. Proc. Natl. Acad. Sci. U.S.A. 101 (33): 12288-93. DOI:10.1073/pnas.0404041101. PMC 514471. PMID 15284440. www.pubmedcentral.nih gov/articlerender.fcgi?tool=pmcentrez&artid=514471.

100. Banaszynski L A, Liu C W, Wandless T J (April 2005). “Characterization of the FKBP.rapamycin.FRB ternary complex”. J. Am. Chem. Soc. 127 (13): 4715-21. DOI:10.1021/ja043277y. PMID 15796538.

101. Sabers C J, Martin M M, Brunn G J, Williams J M, Dumont F J, Wiederrecht G, Abraham R T (January 1995). “Isolation of a protein target of the FKBP12-rapamycin complex in mammalian cells”. J. Biol. Chem. 270 (2): 815-22. DOI:10.1074/jbc.270.2.815. PMID 7822316.

102. Sabatini D M, Barrow R K, Blackshaw S, Burnett P E, Lai M M, Field M E, Bahr B A, Kirsch J, Betz H, Snyder S H (May 1999). “Interaction of RAFT1 with gephyrin required for rapamycin-sensitive signaling”. Science 284 (5417): 1161-4. DOI:10.1126/science.284.5417.1161. PMID 10325225.

103. Ha S H, Kim D H, Kim I S, Kim J H, Lee M N, Lee H J, Kim J H, Jang S K, Suh P G, Ryu S H (December 2006). “PLD2 forms a functional complex with mTOR/raptor to transduce mitogenic signals”. Cell. Signal. 18 (12): 2283-91. DOI:10.1016/j.cellsig.2006.05.021. PMID 16837165.

104. Frias M A, Thoreen C C, Jaffe J D, Schroder W, Sculley T, Carr S A, Sabatini D M (September 2006). “mSin1 is necessary for Akt/PKB phosphorylation, and its isoforms define three distinct mTORC2s”. Curr. Biol. 16 (18): 1865-70. DOI:10.1016/j.cub.2006.08.001. PMID 16919458.

105. Buerger C, DeVries B, Stambolic V (June 2006). “Localization of Rheb to the endomembrane is critical for its signaling function”. Biochem. Biophys. Res. Commun. 344 (3): 869-80. DOI:10.1016/j.bbrc.2006.03.220. PMID 16631613.

106. Jacinto E, Facchinetti V, Liu D, Soto N, Wei S, Jung S Y, Huang Q, Qin J, Su B (October 2006). “SIN1/MIP1 maintains rictor-mTOR complex integrity and regulates Akt phosphorylation and substrate specificity”. Cell 127 (1): 125-37. DOI:10.1016/j.ce11.2006.08.033. PMID 16962653.

107. McMahon L P, Yue W, Santen R J, Lawrence J C (January 2005). “Farnesylthiosalicylic acid inhibits mammalian target of rapamycin (mTOR) activity both in cells and in vitro by promoting dissociation of the mTOR-raptor complex”. Mol. Endocrinol. 19 (1): 175-83. DOI:10.1210/me.2004-0305. PMID 15459249.

108. Oshiro N, Yoshino K, Hidayat S, Tokunaga C, Hara K, Eguchi S, Avruch J, Yonezawa K (April 2004). “Dissociation of raptor from mTOR is a mechanism of rapamycin-induced inhibition of mTOR function”. Genes Cells 9 (4): 359-66. DOI:10.1111/j.1356-9597.2004.00727.x. PMID 15066126.

109. Kawai S, Enzan H, Hayashi Y, Jin Y L, Guo L M, Miyazaki E, Toi M, Kuroda N, Hiroi M, Saibara T, Nakayama H (July 2003). “Vinculin: a novel marker for quiescent and activated hepatic stellate cells in human and rat livers”. Virchows Arch. 443 (1): 78-86. DOI:10.1007/s00428-003-0804-4. PMID 12719976.

110. Choi K M, McMahon L P, Lawrence J C (May 2003). “Two motifs in the translational repressor PHAS-I required for efficient phosphorylation by mammalian target of rapamycin and for recognition by raptor”. J. Biol. Chem. 278 (22): 19667-73. DOI:10.1074/jbc.M301142200. PMID 12665511.

111. Nojima H, Tokunaga C, Eguchi S, Oshiro N, Hidayat S, Yoshino K, Hara K, Tanaka N, Avruch J, Yonezawa K (May 2003). “The mammalian target of rapamycin (mTOR) partner, raptor, binds the mTOR substrates p70 S6 kinase and 4E-BP1 through their TOR signaling (TOS) motif”. J. Biol. Chem. 278 (18): 15461-4. DOI:10.1074/jbc.C200665200. PMID 12604610.

112. Schieke S M, Phillips D, McCoy J P, Aponte A M, Shen R F, Balaban R S, Finkel T (September 2006). “The mammalian target of rapamycin (mTOR) pathway regulates mitochondrial oxygen consumption and oxidative capacity”. J. Biol. Chem. 281 (37): 27643-52. DOI:10.1074/jbc.M603536200. PMID 16847060.

113. Sarbassov D D, Ali S M, Sengupta S, Sheen J H, Hsu P P, Bagley A F, Markhard A L, Sabatini D M (April 2006). “Prolonged rapamycin treatment inhibits mTORC2 assembly and Akt/PKB”. Mol. Cell 22 (2): 159-68. DOI:10.1016/j.molce1.2006.03.029. PMID 16603397.

114. Tzatsos A, Kandror K V (January 2006). “Nutrients suppress phosphatidylinositol 3-kinase/Akt signaling via raptor-dependent mTOR-mediated insulin receptor substrate 1 phosphorylation”. Mol. Cell. Biol. 26 (1): 63-76. DOI:10.1128/MCB.26.1.63-76.2006. PMC 1317643. PMID 16354680. www.pubmedcentral.nih gov/articlerender.fcgi?tool=pmcentrez&artid=1317643.

115. Sarbassov D D, Sabatini D M (November 2005). “Redox regulation of the nutrient-sensitive raptor-mTOR pathway and complex”. J. Biol. Chem. 280 (47): 39505-9. DOI:10.1074/jbc.M506096200. PMID 16183647.

116. Yang Q, Inoki K, Ikenoue T, Guan K L (October 2006). “Identification of Sin1 as an essential TORC2 component required for complex formation and kinase activity”. Genes Dev. 20 (20): 2820-32. DOI:10.1101/gad.1461206. PMC 1619946. PMID 17043309. www.pubmedcentral.nih gov/articlerender.fcgi?tool=pmcentrez&artid=1619946.

117. Chiang G G, Abraham R T (July 2005). “Phosphorylation of mammalian target of rapamycin (mTOR) at Ser-2448 is mediated by p70S6 kinase”. J. Biol. Chem. 280 (27): 25485-90. DOI:10.1074/jbc.M501707200. PMID 15899889.

118. Holz M K, Blenis J (July 2005). “Identification of S6 kinase 1 as a novel mammalian target of rapamycin (mTOR)-phosphorylating kinase”. J. Biol. Chem. 280 (28): 26089-93. DOI:10.1074/jbc.M504045200. PMID 15905173.

119. Isotani S, Hara K, Tokunaga C, Inoue H, Avruch J, Yonezawa K (November 1999) “Immunopurified mammalian target of rapamycin phosphorylates and activates p70 S6 kinase alpha in vitro”. J. Biol. Chem. 274 (48): 34493-8. DOI:10.1074/jbc.274.48.34493. PMID 10567431.

120. Toral-Barza L, Zhang W G, Lamison C, Larocque J, Gibbons J, Yu K (June 2005). “Characterization of the cloned full-length and a truncated human target of rapamycin: activity, specificity, and enzyme inhibition as studied by a high capacity assay”. Biochem. Biophys. Res. Commun. 332 (1): 304-10. DOI:10.1016/j.bbrc.2005.04.117. PMID 15896331.

121. Ali S M, Sabatini D M (May 2005). “Structure of S6 kinase 1 determines whether raptor-mTOR or rictor-mTOR phosphorylates its hydrophobic motif site”. J. Biol. Chem. 280 (20): 19445-8. DOI:10.1074/jbc.C500125200. PMID 15809305.

122. Saitoh M, Pullen N, Brennan P, Cantrell D, Dennis P B, Thomas G (May 2002). “Regulation of an activated S6 kinase 1 variant reveals a novel mammalian target of rapamycin phosphorylation site”. J. Biol. Chem. 277 (22): 20104-12. DOI:10.1074/jbc.M201745200. PMID 11914378.

123. Edinger A L, Linardic C M, Chiang G G, Thompson C B, Abraham R T (December 2003). “Differential effects of rapamycin on mammalian target of rapamycin signaling functions in mammalian cells”. Cancer Res. 63 (23): 8451-60. PMID 14679009.

124. Leone M, Crowell K J, Chen J, Jung D, Chiang G G, Sareth S, Abraham R T, Pellecchia M (August 2006). “The FRB domain of mTOR: NMR solution structure and inhibitor design”. Biochemistry 45 (34): 10294-302. DOI:10.1021/bi060976. PMID 16922504.

125. Kumar V, Pandey P, Sabatini D, Kumar M, Majumder P K, Bharti A, Carmichael G, Kufe D, Kharbanda S (March 2000). “Functional interaction between RAFT1/FRAP/mTOR and protein kinase cdelta in the regulation of cap-dependent initiation of translation”. EMBO J. 19 (5): 1087-97. DOI:10.1093/emboj/19.5.1087. PMC 305647. PMID 10698949. www.pubmedcentral.nih gov/articlerender.fcgi?tool=pmcentrez&artid=305647.

126. Long X, Ortiz-Vega S, Lin Y, Avruch J (June 2005). “Rheb binding to mammalian target of rapamycin (mTOR) is regulated by amino acid sufficiency”. J. Biol. Chem. 280 (25): 23433-6. DOI:10.1074/jbc.C500169200. PMID 15878852.

127. Smith E M, Finn S G, Tee A R, Browne G J, Proud C G (May 2005). “The tuberous sclerosis protein TSC2 is not required for the regulation of the mammalian target of rapamycin by amino acids and certain cellular stresses”. J. Biol. Chem. 280 (19): 18717-27. DOI:10.1074/jbc.M414499200. PMID 15772076.

128. Bernardi R, Guernah I, Jin D, Grisendi S, Alimonti A, Teruya-Feldstein J, Cordon-Cardo C, Simon M C, Rafii S, Pandolfi P P (August 2006). “PML inhibits HIF-1alpha translation and neoangiogenesis through repression of mTOR”. Nature 442 (7104): 779-85. DOI:10.1038/nature05029. PMID 16915281.

129. Kristof A S, Marks-Konczalik J, Billings E, Moss J (September 2003). “Stimulation of signal transducer and activator of transcription-1 (STAT1)-dependent gene transcription by lipopolysaccharide and interferon-gamma is regulated by mammalian target of rapamycin”. J. Biol. Chem. 278 (36): 33637-44. DOI:10.1074/jbc.M301053200. PMID 12807916.

130. Yokogami K, Wakisaka S, Avruch J, Reeves S A (January 2000). “Serine phosphorylation and maximal activation of STATS during CNTF signaling is mediated by the rapamycin target mTOR”. Curr. Biol. 10 (1): 47-50. DOI:10.1016/S0960-9822(99)00268-7. PMID 10660304.

131. Kusaba H, Ghosh P, Derin R, Buchholz M, Sasaki C, Madara K, Longo D L (January 2005). “Interleukin-12-induced interferon-gamma production by human peripheral blood T cells is regulated by mammalian target of rapamycin (mTOR)”. J. Biol. Chem. 280 (2): 1037-43. DOI:10.1074/jbc.M405204200. PMID 15522880.

132. Wu S, Mikhailov A, Kallo-Hosein H, Hara K, Yonezawa K, Avruch J (January 2002). “Characterization of ubiquilin 1, an mTOR-interacting protein”. Biochim. Biophys. Acta 1542 (1-3): 41-56. DOI:10.1016/S0167-4889(01)00164-1. PMID 11853878.

133. Huang S, Houghton P J (2002). “Mechanisms of resistance to rapamycins”. Drug Resist. Updat. 4 (6): 378-91. DOI:10.1054/drup.2002.0227. PMID 12030785.

134. Harris T E, Lawrence J C (2004). “TOR signaling”. Sci. STKE 2003 (212): re15. DOI:10.1126/stke.2122003re15. PMID 14668532.

135. Easton J B, Houghton P J (2005). “Therapeutic potential of target of rapamycin inhibitors”. Expert Opin. Ther. Targets 8 (6): 551-64. DOI:10.1517/14728222.8.6.551. PMID 15584862.

136. Deldicque L, Theisen D, Francaux M (2005). “Regulation of mTOR by amino acids and resistance exercise in skeletal muscle”. Eur. J. Appl. Physiol. 94 (1-2): 1-10. DOI:10.1007/s00421-004-1255-6. PMID 15702344.

137. Weimbs T (2007). “Regulation of mTOR by polycystin-1: is polycystic kidney disease a case of futile repair?”. Cell Cycle 5 (21): 2425-9. DOI:10.4161/cc.5.21.3408. PMID 17102641.

138. Sun S Y, Fu H, Khuri F R (2007). “Targeting mTOR signaling for lung cancer therapy”. Journal of thoracic oncology: official publication of the International Association for the Study of Lung Cancer 1 (2): 109-11. PMID 17409838.

139. Abraham R T, Gibbons J J (2007). “The mammalian target of rapamycin signaling pathway: twists and turns in the road to cancer therapy”. Clin. Cancer Res. 13 (11): 3109-14. DOI:10.1158/1078-0432.CCR-06-2798. PMID 17545512.

140. www.nlm.nih.gov/cgi/mesh/2011/MB_cgi?mode=?term=mTOR+protein

141. Mizgerd, J. P. Lung infection—a public health priority PLoS. Med. 3, e76 (2006).

142. Mizgerd, J. P. Acute lower respiratory tract infection N. Engl. J. Med. 358, 716-727 (2008).

143. Sahly, H. & Podschun, R. Clinical, bacteriological, and serological aspects of Klebsiella infections and their spondylarthropathic sequelae. Clin. Diagn. Lab Immunol 4, 393-399 (1997).

144. Greenberger, M. J. et al. IL-12 gene therapy protects mice in lethal Klebsiella pneumonia. J. Immunol. 157, 3006-3012 (1996).

145. Standiford, T. J. et al. Intrapulmonary tumor necrosis factor gene therapy increases bacterial clearance and survival in murine gram-negative pneumonia. Hum. Gene Ther. 10, 899-909 (1999).

146. Ye, P. et al. Interleukin-17 and lung host defense against Klebsiella pneumoniae infection. Am. J. Respir. Cell Mol. Biol. 25, 335-340 (2001).

147. Cai, S., Batra, S., Shen, L., Wakamatsu, N., & Jeyaseelan, S. Both TRIF- and MyD88-dependent signaling contribute to host defense against pulmonary Klebsiella infection. J. Immunol. 183, 6629-6638 (2009).

148. Schurr, J. R. et al. Central role of toll-like receptor 4 signaling and host defense in experimental pneumonia caused by Gram-negative bacteria. Infect. Immun 73, 532-545 (2005).

149. Moranta, D. et al. Klebsiella pneumoniae capsule polysaccharide impedes the expression of b-defensins by airway epithelial cells. Infect. Immun. 78, 1135-1146 (2010).

150. Regueiro, V., Campos, M. A., Pons, J., Alberti, S., & Bengoechea, J. A. The uptake of a Klebsiella pneumoniae capsule polysaccharide mutant triggers an inflammatory response by human airway epithelial cells. Microbiology 152, 555-566 (2006).

151. Lawlor, M. S., Handley, S. A., & Miller, V. L. Comparison of the host responses to wild-type and cpsB mutant Klebsiella pneumoniae infections. Infect. Immun. 74, 5402-5407 (2006).

152. Regueiro, V. et al. Klebsiella pneumoniae subverts the activation of inflammatory responses in a NOD1-dependent manner. Cell Microbiol. 13, 135-153 (2011).

153. Saitoh, T. & Akira, S. Regulation of innate immune responses by autophagy-related proteins J. Cell Biol. 189, 925-935 (2010).

154. Deretic, V. & Levine, B. Autophagy, immunity, and microbial adaptations Cell Host. Microbe 5, 527-549 (2009).

155. Yoshida, K. et al. Induction of interleukin-10 and down-regulation of cytokine production by Klebsiella pneumoniae capsule in mice with pulmonary infection. J. Med. Microbiol. 50, 456-461 (2001).

156. O'Brien A. D., Standiford T. J., Bucknell K. A., Wilcoxen S. E., Paine R. 3rd, “Role of alveolar epithelial cell intercellular adhesion molecule-1 in host defense against Klebsiella pneumoniae”, Am J Physiol.; 276(6 Pt 1):L961-70 (1999).

157. Sutherland, R. E., Olsen, J. S., McKinstry, A., Villalta, A., Wolters, P. J., “Killing Pneumonia and Sepsis by Enhancing Neutrophil Mast Cell IL-6 Improves Survival from Klebsiella”, J Immunol; 181;5598-5605 (2008), www.jimmunol.org/content/181/8/5598.full.pdf.

TABLE 1
Kinases validated

| Name | Description |
|---|---|
| CDK5 | cyclin-dependent kinase 5 |
| CDK8 | cyclin-dependent kinase 8 |
| CDK9 | cyclin-dependent kinase 9 (CDC2-related kinase) |
| CIB3 | calcium and integrin binding family member 3 |
| CKB | creatine kinase, brain |
| DAPK2 | death-associated protein kinase 2 |
| DUSP24 | dual-specificity tyrosine-(Y)-phosphorylation regulated |
| | kinase 1A |
| GSK3A | glycogen synthase kinase 3 alpha |
| HGS | hepatocyte growth factor-regulated tyrosine kinase substrate |
| HIPK1 | homeodomain interacting protein kinase 1 |
| ILK | integrin-linked kinase |
| ERK | mitogen-activated protein kinase 3 |
| PAK4 | p21(CDKN1A)-activated kinase 4 |
| PCTK3 | PCTAIRE protein kinase 3 |
| PIK3AP1 | Phosphoinositide-3-kinase adaptor protein 1 |
| PIK3R1 | Phosphoinositide-3-kinase, regulatory subunit, |
| | polypeptide 1 (p85 alpha) |
| PKMYT1 | membrane-associated tyrosine- and |
| | threonine-specific cdc2-inhibitory kinase |
| PRKACB | protein kinase, cAMP-dependent, catalytic, beta |
| PRKCABP | protein kinase C, alpha binding protein |
| ROR2 | receptor tyrosine kinase-like orphan receptor 2 |
| SRPK1 | SFRS protein kinase 1 |
| TTK | TTK protein kinase |
| TYRO3 | TYRO3 protein tyrosine kinase |
| UCK1 | uridine-cytidine kinase 1 |

What is claimed is:

1. A method for treating a mammal having an infection with an organism that persists intracellularly in a mammalian cell by at least one of reducing autophagy, inducing elevated levels of anti-inflammatory cytokines, and reducing levels of inflammatory cytokines, comprising administering to the mammal an therapeutic dose of a bioavailable agent which effectively increases autophagy function, by inhibition of at least one element within the mTOR pathway.

2. The method according to claim 1, wherein the bioavailable agent inhibits mTOR.

3. The method according to claim 1, wherein the bioavailable agent inhibits a protein kinase.

4. The method according to claim 1, wherein the bioavailable agent comprises rapamycin.

5. The method according to claim 1, wherein the bioavailable agent comprises a selective PI-3K inhibitor.

6. The method according to claim 1, wherein the bioavailable agent comprises comprising a selective EGFR inhibitor.

7. The method according to claim 1, wherein the bioavailable agent is effective for restoring an NF-κB activation as part of a physiological inflammatory response in airway epithelial cells and alveolar macrophages which is selectively inhibited by Klebsiella pneumoniae infection.

8. The method according to claim 1, further comprising concurrently administering a broad-spectrum antibiotic.

9. The method according to claim 8, wherein the bioavailable agent and the broad spectrum antibiotic are provided in a pharmaceutically acceptable dosage form comprising an oral dosage form configured to provide an effective dose of the bioavailable agent and the broad spectrum antibiotic, and are administered in a course of therapy comprising 1 to 4 oral doses per day.

10. A method of treating a mammal persistently infected with Klebsiella pneumoniae, comprising administering an effective amount in an effective regimen of an mTOR inhibitor to increase autophagocytic function of lung macrophages to kill the Klebsiella pneumoniae.

11. The method according to claim 10, wherein the mTOR inhibitor is provided in a pharmaceutically acceptable, orally bioavailable dosage form adapted for administration to an adult human in an efficacious dose.

12. The method according to claim 10, wherein the bioavailable agent comprises rapamycin.

13. A method of enhancing macrophage response in a subject having an infection with an intracellular organism that activates mTOR, in need of treatment, comprising administering to the subject a therapeutically effective amount of an mTOR inhibitor, thereby inhibiting mTOR and enhancing autophagocytic activity of the macrophage.

14. The method according to claim 13, wherein the mTOR inhibitor is provided in an efficacious dose in a pharmaceutically acceptable, orally bioavailable dosage form adapted for administration to an adult human.

15. The method according to claim 13, wherein the bioavailable agent comprises rapamycin.

16. A pharmaceutically acceptable dosage form for administration to a human, comprising: a pharmaceutically acceptable inhibitor of an effect of an mTOR agonist on reducing autophagocytosis of a bacteria living within an autophagosome, in an effective amount and bioavailable form to increase an autophagocytic function of a mammalian cell to kill the bacteria.

17. The pharmaceutically acceptable dosage form according to claim 16, wherein the pharmaceutically acceptable inhibitor of an effect of an mTOR agonist comprises an mTOR inhibitor.

18. The pharmaceutically acceptable dosage form according to claim 16, wherein the pharmaceutically acceptable inhibitor of an effect of an mTOR agonist comprises a protein kinase inhibitor that is adapted to achieve an effective level in adult human lung tissue.

19. The pharmaceutically acceptable dosage form according to claim 16, wherein the pharmaceutically acceptable inhibitor of an effect of an mTOR agonist comprises rapamycin.

20. The pharmaceutically acceptable dosage form according to claim 16, comprising a selective PI-3K inhibitor.

21. The pharmaceutically acceptable dosage form according to claim 16, comprising a selective EGFR inhibitor.

22. The pharmaceutically acceptable dosage form according to claim 16, wherein the pharmaceutically acceptable inhibitor of an effect of an mTOR agonist is effective for restoring an NF-κB activation as part of a physiological inflammatory response in airway epithelial cells and alveolar macrophages which is selectively inhibited by Klebsiella pneumoniae infection.

23. The pharmaceutically acceptable dosage form according to claim 16, further comprising a broad-spectrum antibiotic.

24. The pharmaceutically acceptable dosage form according to claim 23, wherein the pharmaceutically acceptable dosage form comprises an oral dosage form configured to provide an effective dose of the pharmaceutically acceptable inhibitor of an effect of an mTOR agonist and the broad spectrum antibiotic in a course of therapy comprising 1 to 4 oral doses per day.
